(19) United States
(12) Patent Application Publication
(10) Pub. No.: US 2010/0279316 A1
(43) Pub. Date: Nov. 4, 2010

(54) Antibodies to Phosphorylated IRAK4

(76) Inventors: Leonid Gorelik, Quincy, MA (US); Darren P. Baker, Hingham, MA (US); Catherine A. Hession, Hingham, MA (US); Karyn Van De Mark, Woburn, MI (US); Robert M. Arduini, Charlestown, MA (US); Craig Wildes, Cambridge, MA (US)

Correspondence Address: STERNE, KESSLER, GOLDSTEIN & FOX, P.L.L.C., 1100 NEW YORK AVE., N.W., WASHINGTON, DC, 20005, US

(21) Appl. No.: 12/523,681

(22) PCT Filed: Jan. 9, 2008
(86) PCT No.: PCT/US08/00316

§ 371 Date: Jul. 1, 2010

Related U.S. Application Data
(60) Provisional application No. 60/881,190, filed on Jan. 19, 2007.

(51) Int. Cl. G01N 33/567; C07K 16/40; C12N 5/12; G01N 33/566

(52) U.S. Cl. 435/7.21; 530/387.9; 435/346; 435/7.1; 530/387.3

(57) ABSTRACT

The present invention relates to antibodies that bind phosphorylated forms of IRAK4, methods of using such antibodies to detect IRAK4 biological activity, and methods for the detection, diagnosis, and prognostication of pathological conditions related to IRAK4 biological activity. ERAK4 is one member of a small family of highly conserved cytoplasmic signal transduction proteins characterized by the presence of an N-terminal “death domain” and a C-terminal serine-threonine kinase domain. IRAK4 functions in cytoplasmic signal transduction pathways by interacting with membrane spanning proteins which play, inter alia, critical roles in vertebrate immune system function.

### FIELD OF THE INVENTION

This invention relates to immune diseases and disorders such as, but not limited to, those involving inflammation, autoimmune disorders, and recurrent bacterial infections. More particularly, this invention relates to antibodies useful for detecting, diagnosing, and prognosticating immune diseases and disorders, as well as for identifying compounds capable of interacting with the protein to which said antibodies bind.

### BACKGROUND OF THE INVENTION

Cellular immune responses depend on the ability of immune cells (e.g., macrophages, natural killer cells, T-cells) to detect and respond to cues in the extracellular environment by transmitting (transducing) signals across the cell membrane and into the intracellular (cytoplasmic) environment. Signals transmitted across the cell membrane may then effect a variety of “downstream” cytoplasmic and nuclear signal transduction pathways that subsequently produce a variety of immune cell responses (for example up- or down-regulation of gene transcription and translation or by releasing cytoplasmically stored components into the extracellular environment).

One cytoplasmic molecule responsible for the transmission of such downstream signals is known as “IRAK4”. IRAK4 belongs to a small family of highly conserved cytoplasmic signal transduction proteins. See, Lasker et al., Molecular structure of the IL-1R-Associated Kinase-4 Death Domain and its implications for TLR signaling. J. Immunol. 175: 4175-4179 (2005); see also, Li, et al., IRAK-4: A novel member of the IRAK family with the properties of an IRAK-kinase., Proc. Natl. Acad. Sci. USA 99: 5567-5572 (2002). Currently, at least four members of the IRAK family of proteins are known to exist: IRAK1 (see, e.g., Cao, Z., et al., IRAK: A Kinase Associated with the Interleukin-1 Receptor. Science, 271: 1128-1131 (1996)); IRAK2 (see, e.g., Muzio, M., et al., IRAK (Pelle) Family Member IRAK-2 and MyD88 as Proximal Mediators of IL-1 Signalling. Science, 278: 1612-1615 (1997)); IRAK-M (see, e.g., Wesche, H., et al., IRAK-M Is a Novel Member of the Pelle/Interleukin-1 Receptor-associated Kinase (IRAK) Family. J. Biol. Chem., 274: 19403-19410 (1999); and, IRAK4 (see, e.g., Li, S., et al., IRAK4: A novel member of the IRAK family with the properties of an IRAK-kinase. Proc. Natl. Acad. Sci., 99: 5567-5572 (2002)). IRAK proteins are typified by the presence of an N-terminal “death domain” and a C-terminal serine-threonine kinase domain. Of the four known IRAK proteins, only IRAK1 and IRAK4 are active as serine-threonine kinases.

IRAK4 functions in cytoplasmic signal transduction pathways by interacting with components (“adaptor proteins”) associated with the cytoplasmic portion of the Interleukin-1 receptor (IL-1R), Interleukin-18 receptor (IL-18R), and Toll-Like receptors (TLRs). These receptors (ILRs and the vertebrate TLRs) play critical roles in innate immunity (i.e., general, non-specific immune system mechanisms of defense). In particular, TLRs play critical roles in responding to microbial pathogens. TLRs are capable of eliciting a generalized immune response to pathogens via recognition of pathogen-associated molecular patterns (PAMPs). In response to such PAMPs, IL-1R/TLR signal transduction is initiated, across the cell membrane, by recruiting cytoplasmic adaptor proteins. Such adaptor proteins interact with homologous Toll/IL-1R (TIR) domains located in the cytoplasmic portion of IL-1R/TLR receptors. See, Takeda, et al., Toll-Like Receptors, Ann. Rev. Immunol., vol. 21: pp. 335-376 (2003); Medzhitov & Janeway, Innate immunity, N. Engl. J. Med., vol. 343: pp. 338-344 (2000); and, Imler & Hoffmann, Toll and Toll-like proteins: an ancient family of receptors signaling infection. Rev. Immunogenet. 2:294-304 (2000)).

The importance of adaptor proteins to immune system function is well established, as elimination of such adaptor proteins has been shown to induce significant disruptions of innate immune responses. See, Janssens & Beyaert, Functional diversity and regulation of different interleukin-1 receptor-associated kinase (IRAK) family members, Mol. Cell., 11:293-302 (2003). Some examples of known IL-1R/TLR adaptor proteins are: MyD88; TIRAP/Mal; Trif/Ticam; and TRAM. See, O'Neill, et al., The Toll-IL-1 receptor adaptor family grows to five members. Trends Immunol. 24:286-290 (2003). MyD88, in particular, has a modular “death domain” (DD) that functions to recruit IRAK family proteins such as IRAK4. See, Burns et al., Inhibition of interleukin 1 receptor/Toll-like receptor signaling through the alternatively spliced short form of MyD88 is due to its failure to recruit IRAK-4, J. Exp. Med. 197: 263-268 (2003). IRAK4 is thought to associate with MyD88 via IRAK4's own death domain. Moreover, loss of IRAK4/MyD88 association disrupts IL-1R/TLR signal transduction by preventing IRAK4 from phosphorylating (i.e., activating) IRAK1. See, Janssens et al. and Burns et al.

IRAK4 Signal Transduction Pathways: In response to the appropriate external cues, IL-1R/TLRs form a ligand-induced receptor complex which can then recruit MyD88 via hydrophilic interaction of mutual TIR domains. See e.g., Janssens & Beyaert. MyD88 also interacts with the IRAK family proteins, IRAK1 and IRAK4. These latter interactions facilitate phosphorylation of IRAK1 by IRAK4. IRAK1 subsequently interacts with TNFR-associated factor 6 (TRAF6). Thereafter, the IRAK-1/TRAF-6 complex dissociates from the receptor, whereupon it interacts with the kinase TAK1 and two TAK-1 binding proteins, TAB-1 and TAB-2, at the plasma membrane. See, Takaesu, G., et al., TAB2, a novel adaptor protein, mediates activation of TAK1 MAPKKK by linking TAK1 to TRAF6 in the IL-1 signal transduction pathway, Mol. Cell., 5: 649-658 (2000); and Jiang, Z., et al., Interleukin-1 (IL-1) receptor-associated kinase-dependent IL-1-induced signaling complexes phosphorylate TAK1 and TAB2 at the plasma membrane and activate TAK1 in the cytosol. Mol. Cell. Biol., 22: 7158-7167 (2002).

IRAK-1 is subsequently released and degraded in a ubiquitin-dependent manner. A TRAF-6/TAK-1/TAB-1/TAB-2 complex then dissociates from the plasma membrane where it leads to the activation of the IKK complex. The activated IKK complex then phosphorylates IκB, resulting in the nuclear translocation of NF-κB and upregulation of gene transcription See Cao, Z., Henzel, W. J. and Gao, X., IRAK: A kinase associated with the Interleukin-1 receptor, Science, 271: 1128-1131 (1996).

TRAF6 also mediates MAP kinase (MAPK) activation which leads to upregulation of gene expression via activation of AP-1 transcription factors. See, Burns, K., et al., Inhibition of Interleukin 1 Receptor/Toll-like Receptor Signalling through the Alternatively Spliced, Short Form of MyD88 Is Due to Its Failure to Recruit IRAK4., J. Exp. Med., 197: 263-268 (2003); Wesche, H., et al., MyD88, An Adaptor That Recruits IRAK to the IL-1 Receptor Complex. Immunity, 7: 837-847 (1997); and, Cao, Z., W. J. Henzel, and X. Gao, IRAK: A Kinase Associated with the Interleukin-1 Receptor. Science, 271: 1128-1131 (1996).

Biologically, IRAK4 has been demonstrated to play a critical role in innate immunity. For example, a deficiency of IRAK4 in humans results in recurrent pyogenic bacterial infections. See, Picard et al., Pyogenic bacterial infections in humans with IRAK-4 deficiency, Science, 299, 2076-2079 (2003); and, Medvedev et al. Distinct mutations in IRAK-4 confer hypo-responsiveness to lipopolysaccharide and interleukin-1 in a patient with recurrent bacterial infections, J. Exp. Med., 198, 521-531 (2003). Indeed, at least 18 individuals from 11 unrelated families have been identified as deficient in IRAK4. See, Turvey, et al., Towards subtlety: Understanding the role of Toll-like receptor signaling in susceptibility to human infections. Clinical Immunol., vol. 120, pp. 1-9 (2006). Furthermore, distinct mutations in IRAK-4 confer hypo-responsiveness to lipopolysaccharide and interleukin-1 in a patient with recurrent bacterial infections, J. Exp. Med., vol. 198, no. 4: pp. 521-531 (2003). Indeed, in at least one such patient the IRAK4 deficiency resulted from point mutations wherein the IRAK4 protein had an intact DD but a truncated kinase domain. See, Medvedev et al. Additionally, cells from patients deficient in IRAK4 function were unable to activate the NFκB pathway and were unable to produce inflammatory cytokines in response to TLR-, IL-1-, or IL-18-mediated signals. See, Ku, C.-L., et al., Inherited disorders of human Toll-like receptor signalling: Immunological implications. Immunol. Rev., 203: 10-20 (2005). For example, patients with IRAK4 deficiency have been found to be lacking in the ability to produce TNF-α, IL-6, IFN-γ, IL-1b, IL-6, IL-8, and IL-12 in response to known TLR agonists. See, Ku et al., Inherited disorders of human Toll-like receptor signaling: Immunological implications. Immunological Reviews, vol. 203, pp. 10-20 (2005). Likewise, investigators have also demonstrated that mice lacking IRAK4 are resistant to lethal dose administration of lipopolysacharide (a bacterial endotoxin) and are significantly impaired in responding to other bacterial and viral challenges. See, Suzuki, et al., Severe impairment of interleukin-1 and Toll-like receptor signalling in mice lacking IRAK4, Nature, vol. 416, pp. 750-756 (2002).

While it is clear that IRAK4 plays a critical role in the innate immune system, the importance of its kinase activity has been the subject of some controversy. Lye et al. showed that the kinase activity of IRAK4 was important for its signaling function, while Qin et al. showed that it was dispensable. See, Lye, E. et al., The Role of Interleukin 1 Receptor-Associated Kinase-4 (IRAK4) Kinase Activity in IRAK-4-Mediated Signaling. J. Biol. Chem., 279: 40653-40658 (2004); and, Qin, J., et al., IRAK4 Kinase Activity Is Redundant for Interleukin-1 (IL-1) Receptor-associated Kinase Phosphorylation and IL-1 Responsiveness. J. Biol. Chem., 2004. 279: 26748-26753 (2004). However, it is important to note that both these studies were performed with transfected cells in vitro, and, therefore, may not reflect the situation found in vivo where IRAK4 is expressed at physiological levels. To address this, Koziczak-Holbro et al. more recently demonstrated that “knock-in” mice (having an endogenous gene replaced with an altered gene), in which a gene encoding full-length wild type IRAK4 was replaced with a gene encoding IRAK4 containing inactivating mutations in the kinase domain (K213A/K214A; referred to herein as kinase inactive IRAK4), were unable to mount normal responses to IL-1β. See, Koziczak-Holbro, M., et al., The Role of IRAK4 Kinase Activity in IL-1Beta-Mediated Signaling. Eur. Cytokine Netw., 17: 31-32 (2006).

In embryonic fibroblasts from these knock-in mice (MEFs), IL-1 β failed to activate the NF-κB, p38, and JNK signal transduction pathways. Furthermore, in bone marrow-derived macrophages from wild type, IRAK4 kinase inactive, and IRAK4 knockout mice (in which the entire IRAK4 protein was deleted), levels of IRAK1 protein were stabilized in the kinase inactive and IRAK4 knockout MEFs, as compared to MEFs with wild-type IRAK4. This suggests that the absence of kinase activity in the IRAK4 kinase inactive cells (and in the knockout cells) prevents IRAK4-dependent phosphorylation of IRAK1. In turn, TRAF6 does not associate with IRAK1 and is not delivered to the plasma membrane where it normally associates with TAK-1, TAB-1, and TAB-2. As IRAK1 is ubiquinated and degraded following release of TRAF6, the absence of IRAK4 kinase activity results in the stabilization of the levels of IRAK1. The role of the kinase activity of IRAK4 was also assessed in a foot pad swelling model in wild type mice and mice with kinase inactive IRAK4. In contrast to the wild type mice which showed significant swelling, mice with kinase inactive IRAK4 showed little or no swelling. Together these data indicate that in a mouse model system, IRAK4 kinase activity plays a critical role in mediating IL-1-dependent signals in vivo.

Considering the importance of IRAK4 in regulating immune system responses, the ability to detect, diagnose, and modulate both normal and aberrant IRAK4 biological activities would provide an important avenue of information, intervention, and treatment for immune system disorders in which IRAK4 is a participant. Additionally, the ability to readily identify compounds that enhance or inhibit IRAK4 biological activities would also provide an important means for discovering and developing additional compounds affecting such functions.

### BRIEF SUMMARY OF THE INVENTION

Thus, the present invention is based on the discovery that phosphorylation of amino acid residues in the IRAK4 kinase domain (i.e., spanning residues Thr-163 to Ser-460 of the full-length protein) is essential to IRAK4 biological activities. See, SEQ ID NO:1 (IRAK4 kinase domain) and SEQ ID NO:2 (full-length IRAK4); see also, National Center for Biotechnology Information (NCBI) Protein Database (www.ncbi.nlm.nih.gov); Accession No. NP_{—}057207 for human IRAK4 sequence. Accordingly, here we describe the cloning, expression, and purification of the kinase domain of human IRAK4, the identification of Thr-342, Thr-345, and Ser-346 as targets of auto/transphosphorylation, and the development of antibodies that specifically bind IRAK4 epitopes comprising phosphorylated amino acid residues.

Moreover, it has been discovered that in vitro kinase assays performed with the IRAK4 kinase domain produce greater than 95% phosphorylation of amino acid residues Thr-342, Thr-345, and Ser-346; each of which are in the activation loop of the IRAK4 kinase domain. (It has also been discovered that these same in vitro kinase assays produce approximately 30% phosphorylation of amino acid residues Ser-167 and Ser-169.) Furthermore, it has been discovered (as demonstrated by experiments described herein) that phosphorylation of IRAK4 at Thr-342 and Thr-345 is essential for most of IRAK4's kinase activity. Accordingly, as described herein, polyclonal and monoclonal antibodies have been developed that specifically bind to IRAK4 phosphorylated-Thr345. Such antibodies are useful in the detection and diagnosis of pathological conditions (e.g., immune diseases and disorders) in which the kinase activity of IRAK4 is over- or under-activated. Furthermore, such antibodies are also useful for the identification of compounds capable of enhancing or inhibiting IRAK4 kinase activity.

Based on these discoveries, the present invention relates generally to antibodies, antigen binding fragments, or derivatives thereof which can be used to detect and identify phosphorylated (i.e., kinase activated) IRAK4. Additionally, the invention generally relates to methods for identifying additional compounds capable of regulating (inhibiting or enhancing) IRAK4 kinase activity. More particularly, the invention relates to antibodies, antigen binding fragments, or derivatives thereof that specifically bind IRAK4 phosphorylated-Thr345 or IRAK4 phosphorylated-Thr342.

As one exemplary embodiment of the invention, a hybridoma cell line which expresses anti-IRAK4 phospho-Thr345 monoclonal antibody (hybridoma cell line “IRAK4-101C2” or more simply “101C2”) was deposited with the ATCC on Nov. 30, 2006 and given ATCC Patent Deposit Designation PTA-8050. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

The present invention is further directed to isolated polypeptides which make up IRAK4 antibodies, and polynucleotides encoding such polypeptides. IRAK4 antibodies of the present invention comprise polypeptides, e.g., amino acid sequences encoding phosphorylated-IRAK4-specific antigen binding regions derived from immunoglobulin molecules. A polypeptide or amino acid sequence “derived from” a designated protein refers to the origin of the polypeptide. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the present invention provides an isolated polypeptide comprising, consisting essentially of, or consisting of an immunoglobulin heavy chain variable region (VH), where at least one of CDRs of the heavy chain variable region or at least two of the CDRs of the heavy chain variable region are at least 80%, 85%, 90% or 95% identical to reference heavy chain CDR1, CDR2 or CDR3 amino acid sequences from monoclonal IRAK4 antibodies disclosed herein. Alternatively, the CDR1, CDR2 and CDR3 regions of the VH are at least 80%, 85%, 90% or 95% identical to reference heavy chain CDR1, CDR2 and CDR3 amino acid sequences from monoclonal IRAK4 antibodies disclosed herein. In certain embodiments, an antibody or antigen-binding fragment comprising the VH encoded by the polynucleotide specifically or preferentially binds to IRAK4.

In certain embodiments, an antibody or antigen-binding fragment thereof comprising, consisting essentially of, or consisting of a one or more of the VH polypeptides described above specifically or preferentially binds to the same epitope as a reference monoclonal antibody secreted by a hybridoma cell line selected from the group consisting of 101C2, 102C4, 103B6, and 142B2, or will competitively inhibit such a monoclonal antibody from binding to IRAK4.

In certain embodiments, an antibody or antigen-binding fragment thereof comprising, consisting essentially of, or consisting of one or more of the VH polypeptides described above specifically or preferentially binds to an IRAK4 polypeptide or fragment thereof, or a IRAK4 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another embodiment, the present invention provides an isolated polypeptide comprising, consisting essentially of, or consisting of an immunoglobulin light chain variable region (VL), where at least one of the CDRs of the light chain variable region or at least two of the CDRs of the light chain variable region are at least 80%, 85%, 90% or 95% identical to reference heavy chain CDR1, CDR2, or CDR3 amino acid sequences from monoclonal IRAK4 antibodies disclosed herein. Alternatively, the CDR1, CDR2 and CDR3 regions of the VL are at least 80%, 85%, 90% or 95% identical to reference light chain CDR1, CDR2, and CDR3 amino acid sequences from monoclonal IRAK4 antibodies disclosed herein. In certain embodiments, an antibody or antigen-binding fragment comprising the VL polypeptide specifically or preferentially binds to IRAK4.

In certain embodiments, an antibody or antigen-binding fragment thereof comprising, consisting essentially of, one or more of the VL polypeptides described above specifically or preferentially binds to the same epitope as a reference monoclonal antibody secreted by a hybridoma cell line selected from the group consisting of 101C2, 102C4, 103B6, and 142B2, or will competitively inhibit such a monoclonal antibody from binding to IRAK4.

In certain embodiments, an antibody or antigen-binding fragment thereof comprising, consisting essentially of, or consisting of a one or more of the VL polypeptides described above specifically or preferentially binds to an IRAK4 polypeptide or fragment thereof, or a IRAK4 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polypeptides described above may further include additional polypeptides, e.g., a signal peptide to direct secretion of the encoded polypeptide, antibody constant regions as described herein, or other heterologous polypeptides as described herein. Additionally, polypeptides of the invention include polypeptide fragments as described elsewhere. Additionally polypeptides of the invention include fusion polypeptide, Fab fragments, and other derivatives, as described herein.

Also, as described in more detail elsewhere herein, the present invention includes compositions comprising the polypeptides described above.

It will also be understood by one of ordinary skill in the art that IRAK4 antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Certain IRAK4 antibody polypeptides of the present invention comprise, consist essentially of, or consist of an amino acid sequence derived from a human amino acid sequence. However, certain IRAK4 antibody polypeptides comprise one or more contiguous amino acids derived from another mammalian species. For example, an IRAK4 antibody of the present invention may include a primate heavy chain portion, hinge portion, or antigen binding region. In another example, one or more murine-derived amino acids may be present in a non-murine antibody polypeptide, e.g., in an antigen binding site of an IRAK4 antibody. In certain therapeutic applications, IRAK4-specific antibodies, or antigen-binding fragments, variants, or analogs thereof are designed so as to not be immunogenic in the animal to which the antibody is administered.

In certain embodiments, an IRAK4 antibody polypeptide comprises an amino acid sequence or one or more moieties not normally associated with an antibody. Exemplary modifications are described in more detail below. For example, a single-chain fv antibody fragment of the invention may comprise a flexible linker sequence, or may be modified to add a functional moiety (e.g., PEG, a drug, a toxin, or a label).

An IRAK4 antibody polypeptide of the invention may comprise, consist essentially of, or consist of a fusion protein. Fusion proteins are chimeric molecules which comprise, for example, an immunoglobulin antigen-binding domain with at least one target binding site, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an IRAK4 antibody, or an antigen-binding fragment, variant, or analog thereof is derived from a non-immunoglobulin polypeptide of the same species, or an immunoglobulin or non-immunoglobulin polypeptide of a different species.

A “conservative amino acid substitution” is one in which the amino acid residue is replaced with an amino acid residue having a similar side chain. Families of amino acid residues having similar side. chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into IRAK4 antibodies for use in the detection, diagnostic, and/or prognostic methods disclosed herein and screened for their ability to bind to the desired antigen, e.g., phosphorylated-IRAK4.

The present invention is more specifically directed to an IRAK4 antibody, or antigen-binding fragment, variant or derivatives thereof, where the IRAK4 antibody binds to the same phosphorylated epitope as a reference monoclonal antibody secreted by a hybridoma cell line selected from the group consisting of 101C2, 102C4, 103B6, and 142B2.

The invention is further drawn to an IRAK4 antibody, or antigen-binding fragment, variant or derivatives thereof, where the IRAK4 antibody competitively inhibits a monoclonal antibody secreted by a hybridoma cell line selected from the group consisting of 101C2, 102C4, 103B6, and 142B2.

The invention is also drawn to an IRAK4 antibody, or antigen-binding fragment, variant or derivatives thereof, where the IRAK4 antibody comprises at least the antigen binding region of a reference monoclonal antibody secreted by a hybridoma cell line selected from the group consisting of 101C2, 102C4, 103B6, and 142B2.

In certain embodiments, the present invention is directed to an antibody, or antigen-binding fragment, variant, or derivative thereof which specifically or preferentially binds to a phosphorylated-IRAK4 polypeptide fragment or domain. Such phosphorylated-IRAK4 polypeptide fragments include, but are not limited to, an IRAK4 polypeptide comprising, consisting essentially of, or consisting of amino acids Lys-Phe-Ala-Gln-Thr-Val-Met-^{Phos-}Thr-Ser-Arg-Ile-Val-Gly-Thr-Thr (SEQ ID NO:4), where ^{Phos-}Thr signifies a phosphorylated Threonine residue corresponding to phospho-Thr-345 in IRAK4.

In other embodiments, the present invention includes an antibody, or antigen-binding fragment, variant, or derivative thereof which specifically or preferentially binds to at least one phosphorylated epitope of IRAK4, where the epitope comprises, consists essentially of, or consists of at least about four to five amino acids of, at least seven, at least nine, or between at least about 15 to about 30 amino acids of SEQ ID NO:2. The amino acids of a given phosphorylated epitope of SEQ ID NO:2 as described may be, but need not be contiguous or linear. In certain embodiments, the at least one epitope of IRAK4 comprises, consists essentially of, or consists of a non-linear epitope formed by the kinase domain of IRAK4. Thus, in certain embodiments the at least one phosphorylated epitope of IRAK4 comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, where non-contiguous amino acids form an epitope through protein folding.

In certain aspects, the present invention is directed to an antibody, or antigen-binding fragment, variant, or derivative thereof which specifically binds to a phosphorylated-IRAK4 polypeptide or fragment thereof, or a phosphorylated-IRAK4 variant polypeptide, with an affinity characterized by a dissociation constant (K_{D}) which is less than the K_{D} for said reference monoclonal antibody.

In certain embodiments, an antibody, or antigen-binding fragment, variant, or derivative thereof of the invention binds specifically to at least one phosphorylated epitope of IRAK4 or fragment or variant described above, i.e., binds to such a phosphorylated epitope more readily than it would bind to an unrelated, or random epitope; binds preferentially to at least one phosphorylated epitope of IRAK4 or fragment or variant described above, i.e., binds to such a phosphorylated epitope more readily than it would bind to a related, similar, homologous, or analogous epitope; competitively inhibits binding of a reference antibody which itself binds specifically or preferentially to a certain epitope of IRAK4 or fragment or variant described above; or binds to at least one phosphorylated epitope of IRAK4 or fragment or variant described above with an affinity characterized by a dissociation constant K_{D} of less than about 5×10⁻² M, about 10⁻² M, about 5×10⁻³ M, about 10⁻³M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. In a particular aspect, the antibody or fragment thereof preferentially binds to phosphorylated human IRAK4 polypeptide or fragment thereof, relative to phosphorylated murine IRAK4 polypeptide or fragment thereof.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, an antibody, or antigen-binding fragment, variant, or derivative thereof of the invention binds phosphorylated-IRAK4 polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. Alternatively, an antibody, or antigen-binding fragment, variant, or derivative thereof of the invention binds phosphorylated-IRAK4 polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, an antibody, or antigen-binding fragment, variant, or derivative thereof of the invention binds phosphorylated-IRAK4 polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, an antibody, or antigen-binding fragment, variant, or derivative thereof of the invention binds phosphorylated-IRAK4 polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹. The present invention also encompasses polynucleotides and polynucleotide variants encoding the antibodies and antibody fragments described herein.

### BRIEF DESCRIPTION OF THE DRAWINGS/FIGURES

FIG. 1: IRAK4 kinase domain residues threonine-342 (Thr342), threonine-345 (Thr345), and serine-346 (ser346) are targets of in vitro IRAK4 auto-/trans-phosphorylation (see Example D).

FIG. 2: Antibodies specific to phosphosphorylated-Thr345 (P-Thr345) bind to auto-/trans-phosphorylated IRAK4 (see Example E).

FIG. 3: IRAK4 kinase activity is required for auto-/trans-phosphorylation of Thr345 (see Example F).

FIG. 4: IRAK4 kinase activity and amino acid residues Thr342 and Thr345 are essential for effective IRAK4-mediated inhibition of IL1beta induced (NFkB promoter driven) and constitutive (SV40 promoter driven) luciferase expression (see Example G).

FIG. 5: Wild-type IRAK4, but not kinase inactive or IRAK4-T342A/T345A mutants, induces phosphorylation of EIF2alpha (see Example H).

FIG. 6: Wild-type IRAK4, but not kinase inactive or IRAK4-T342A/T345A mutants, inhibits IRAK1 autophosphorylation and IRAK1-induced IkB phosphorylation (see Example I).

FIG. 7: Wild-type IRAK4, but not kinase inactive or IRAK4-T342A/T345A mutants, induce JNK1/2 phosphorylation (see Example J).

### DETAILED DESCRIPTION OF THE INVENTION

### Definitions

It is to be noted that the term “a” or “an” entity refers to one or more of that entity; for example, “an IRAK4 antibody” or “a phosphorylated-IRAK4 antibody” is understood to represent one or more IRAK4 antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are fragments, derivatives, analogs, or variants of the foregoing polypeptides, and any combination thereof. The terms “fragment,” “variant,” “derivative” and “analog” when referring to IRAK4 antibodies or antibody polypeptides of the present invention include any polypeptides which retain at least some of the antigen-binding properties of the corresponding native antibody or polypeptide. Fragments of polypeptides of the present invention include proteolytic fragments, as well as deletion fragments, in addition to specific antibody fragments discussed elsewhere herein. Variants of IRAK4 antibodies and antibody polypeptides of the present invention include fragments as described above, and also polypeptides with altered amino acid sequences due to amino acid substitutions, deletions, or insertions. Variants may occur naturally or be non-naturally occurring Non-naturally occurring variants may be produced using art-known mutagenesis techniques. Variant polypeptides may comprise conservative or non-conservative amino acid substitutions, deletions or additions. Derivatives of IRAK4 antibodies and antibody polypeptides of the present invention, are polypeptides which have been altered so as to exhibit additional features not found on the native polypeptide. Examples include fusion proteins. Variant polypeptides may also be referred to herein as “polypeptide analogs.” As used herein a “derivative” of an IRAK4 antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an IRAK4 antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an IRAK4 antibody or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The present invention is directed to certain IRAK4 antibodies, or antigen-binding fragments, variants, or derivatives thereof. Unless specifically referring to full-sized antibodies such as naturally-occurring antibodies, the term “IRAK4 antibodies” or “phosphorylated-IRAK4 antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “phosphorylate”, “phosphorylated” and “phosphorylation” are used interchangeably herein to describe the addition of a phosphate (PO₄) group (or groups), to one (or more) amino acid residues in a polypeptide chain (such as, for example, the addition of a phosphoryl (PO₃) moiety to the polar R group of an amino acid).

The terms “phosphorylated-IRAK4 antibodies” or “phosphorylated-IRAK4 antibody” indicate the IRAK-4 antigen is phosphorylated.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (C_{L}) and the heavy chain (C_{H}1, C_{H}2 or C_{H}3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the C_{H}3 and C_{L} domains actually comprise the carboxy-terminus of the heavy and light chain, respectively.

As indicated above, the variable region allows the antibody to selectively recognize and specifically bind epitopes on antigens. That is, the V_{L} domain and V_{H} domain, or subset of the complementarity determining regions (CDRs), of an antibody combine to form the variable region that defines a three dimensional antigen binding site. This quaternary antibody structure forms the antigen binding site present at the end of each arm of the Y. More specifically, the antigen binding site is defined by three CDRs on each of the V_{H} and V_{L} chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table I as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions1

| | Kabat | Chothia |
|---|---|---|
| V_{H} CDR1 | 31-35 | 26-32 |
| V_{H} CDR2 | 50-65 | 52-58 |
| V_{H} CDR3 | 95-102 | 95-102 |
| V_{L} CDR1 | 24-34 | 26-32 |
| V_{L} CDR2 | 50-56 | 50-52 |
| V_{L} CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an IRAK4 antibody or antigen-binding fragment, variant, or derivative thereof of the present invention are according to the Kabat numbering system.

In camelid species, the heavy chain variable region, referred to as V_{H}H, forms the entire antigen-binding domain. The main differences between camelid V_{H}H variable regions and those derived from conventional antibodies (V_{H}) include (a) more hydrophobic amino acids in the light chain contact surface of V_{H} as compared to the corresponding region in V_{H}H, (b) a longer CDR3 in V_{H}H, and (c) the frequent occurrence of a disulfide bond between CDR1 and CDR3 in V_{H}H.

As used herein, the term “antigen binding domain” includes a site that specifically binds an epitope on an antigen (e.g., an epitope of IRAK4). The antigen binding domain of an antibody typically includes at least a portion of an immunoglobulin heavy chain variable region and at least a portion of an immunoglobulin light chain variable region. The binding site formed by these variable regions determines the specificity of the antibody.

Unless it is specifically noted, as used herein a “fragment thereof” in reference to an antibody refers to an antigen-binding fragment, i.e., a portion of the antibody which specifically binds to the antigen.

Antibodies or antigen-binding fragments, variants, or derivatives thereof of the invention include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies (including, e.g., anti-Id antibodies to IRAK4 antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains. Antibodies or immunospecific fragments thereof for use in the diagnostic and therapeutic methods disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a C_{H}1 domain, a hinge (e.g., upper, middle, and/or lower hinge region) domain, a C_{H}2 domain, a C_{H}3 domain, or a variant or fragment thereof. For example, a binding polypeptide for use in the invention may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In another embodiment, a polypeptide of the invention comprises a polypeptide chain comprising a C_{H}3 domain. Further, a binding polypeptide for use in the invention may lack at least a portion of a C_{H}2 domain (e.g., all or part of a C_{H}2 domain). As set forth above, it will be understood by one of ordinary skill in the art that these domains (e.g., the heavy chain portions) may be modified such that they vary in amino acid sequence from the naturally occurring immunoglobulin molecule.

In certain IRAK4 antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the invention are not identical. For example, each monomer may comprise a different target binding site, forming, for example, a bispecific antibody.

The heavy chain portions of a binding polypeptide for use in the detection, diagnostic, and/or prognostic methods disclosed herein may be derived from different immunoglobulin molecules. For example, a heavy chain portion of a polypeptide may comprise a C_{H}1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a V_{L} or C_{L} domain.

IRAK4 antibodies, or antigen-binding fragments, variants, or derivatives thereof disclosed herein may be described or specified in terms of the epitope(s) or portion(s) of an antigen, e.g., a target polypeptide (IRAK4) that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or include non-polypeptide elements, e.g., an “epitope” may include post-translationally added elements of a polypeptide such as a phosphate (PO₄) group (e.g., a phosphoryl (PO₃) moiety added to a polar R group of an amino acid).

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitope recognized by IRAK4 antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of IRAK4.

By “specifically binds,” it is meant that the antibody binds to an epitope more readily than it would bind to a random, unrelated epitope or more readily than it would bind to a related, similar, homologous, or analogous epitope. Thus, an antibody which “specifically binds” to a given epitope would more likely bind to that epitope than to a related epitope, even though such an antibody may cross-react (to some degree) with the related epitope. An example of specific binding, with regard to the present invention, would be an antibody that binds a phosphorylated polypeptide epitope more readily than it binds the same polypeptide epitope in its unphosphorylated form. Thus, an antibody which “specifically binds” to a phosphorylated IRAK4 epitope would more likely bind to that epitope than to the same IRAK4 epitope in its unphosphorylated state; even though such antibody may cross-react (to some degree) with the unphosphorylated IRAK4 epitope. Conversely, an antibody which “specifically binds” to an unphosphorylated IRAK4 epitope would more likely bind to that epitope than to the same phosphorylated IRAK4 epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody or antigen-binding fragment, variant, or derivative disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein may be said to bind a target polypeptide disclosed herein or a fragment or variant thereof with an on rate (k(on)) of greater than or equal to 10³ M⁻¹ sec⁻¹, 5×10³ M⁻¹ sec^{31 1}, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As known in the art, “sequence identity” between two polypeptides is determined by comparing the amino acid sequence of one polypeptide to the sequence of a second polypeptide. When discussed herein, whether any particular polypeptide is at least about 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g. , Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

IRAK4 antibodies or antigen-binding fragments, variants or derivatives thereof of the invention may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

IRAK4 antibodies or antigen-binding fragments, variants or derivatives thereof of the invention may also be described or specified in terms of their binding affinity to a phosphorylated polypeptide of the invention. Preferred binding affinities include those with a dissociation constant or Kd less than 5×10⁻² M, 10⁻² M, 5×10⁻³ M, 10⁻³M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

IRAK4 antibodies or antigen-binding fragments, variants or derivatives thereof of the invention may be “multispecific,” e.g., bispecific, trispecific or of greater multi-specificity, meaning that it recognizes and binds to two or more different epitopes present on one or more different antigens (e.g., proteins) at the same time. Thus, whether an IRAK4 antibody is “monospecfic” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in an IRAK4 antibody or binding polypeptide. Each binding domain specifically binds one epitope. When an IRAK4 antibody or binding polypeptide comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “V_{H} domain” includes the amino terminal variable domain of an immunoglobulin heavy chain and the term “C_{H}1 domain” includes the first (most amino terminal) constant region domain of an immunoglobulin heavy chain. The C_{H}1 domain is adjacent to the V_{H} domain and is amino terminal to the hinge region of an immunoglobulin heavy chain molecule.

As used herein the term “C_{H}2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues.

As used herein, the term “hinge region” includes the portion of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the C_{H}1 and C_{L} regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., IRAK4 antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

By “subject” or “individual” or “animal” or “patient” or “mammal,” is meant any subject, particularly a mammalian subject, for whom detection, diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of an IRAK4 antibody” and “an animal in need of treatment” includes subjects, such as mammalian subjects, that would benefit from administration of an IRAK4 antibody used, e.g., for detection of a phosphorylated-IRAK4 polypeptide (e.g., for a diagnostic procedure) with an IRAK4 antibody. As described in more detail herein, the IRAK4 antibody can be used in unconjugated form or can be conjugated, e.g., to a drug, prodrug, or an isotope.

### IRAK4 Biochemistry

The IL-1 Receptor Associated Kinase 4 (IRAK-4) is one of four IRAK family members (IRAK-1, IRAK-2, IRAK-4, and IRAK-M) that play important roles in mammalian host defense and innate immunity. IRAK proteins contain a conserved death domain at the N-terminus and a Serine/Threonine (Ser/Thr) kinase domain at the C-terminus. However, only IRAK-1 and IRAK-4 contain a kinase domain which is enzymically active. In IRAK-2 and IRAK-M, a critical aspartic acid residue is missing from the kinase domain active site.

Full-length IRAK4 is a protein of 460 amino acids, and it is the closest known human homolog to drosophila Pelle. See, Li, et al., IRAK-4: A novel member of the IRAK family with the properties of an IRAK-kinase., Proc. Natl. Acad. Sci. USA 99: 5567-5572 (2002). Endogenous IRAK4 has been demonstrated to interact with IRAK1 and TRAF6 in an IL-1 dependent manner. IRAK4 has also been shown to activate NF-κb as well as mitogen-activated (MAP) kinase pathways. Id. IRAK4 has been shown to phosphorylate IRAK1, whereas IRAK4 dominant negative mutants block IL-1 induced activation and phosphorylation of IRAK1. Id.

Experiments described herein further demonstrate the following:
Phosphorylation of Thr342 and Thr345 in IRAK4 activation loop (through auto- or trans-phosphorylation) is required for IRAK4 kinase activity;
Detection of phosphorylated Thr345 or Thr342 in cells indicates the presence of activated IRAK4 kinase and the IRAK4 signal transduction pathway;
IRAK4 kinase is required for JNK pathway activation;
IRAK4 kinase activity is required for NFkB activation (in contrast, IRAK1 protein, irrespective of its kinase activity, is capable of activating NFkB, IRF, p38 and JNK pathways);
IRAK4 kinase is required for inhibition of NFkB activation by IRAK1 (as a potential negative feedback loop); and,
IRAK4 kinase activates protein translation inhibition/apoptosis pathways by effecting the phosphorylation of eIF2alpha (possibly by activating PKR kinase).

### IRAK4 Antibodies

### Polypeptides

Methods of making antibodies are well known in the art and described herein. Once antibodies to various phosphorylated polypeptide fragments of IRAK4, or to a full-length phosphorylated form of IRAK4, have been produced, determining which amino acids, or epitope, of IRAK4 to which the antibody or antigen binding fragment binds can be determined by epitope mapping protocols as described herein as well as methods known in the art (e.g. double antibody-sandwich ELISA as described in “Chapter 11—Immunology,” Current Protocols in Molecular Biology, Ed. Ausubel et al., v.2, John Wiley & Sons, Inc. (1996)). Additional epitope mapping protocols may be found in Morris, G. Epitope Mapping Protocols, New Jersey: Humana Press (1996), which are both incorporated herein by reference in their entireties. Epitope mapping can also be performed by commercially available means (i.e. ProtoPROBE, Inc. (Milwaukee, Wis.)).

As one exemplary embodiment of the invention, a hybridoma cell line which expresses anti-IRAK4 phospho-Thr345 monoclonal antibody (referred to as hybridoma cell line “IRAK4-101C2” or more simply “101C2”) was deposited with the ATCC on Nov. 30, 2006 and given ATCC Patent Deposit Designation PTA-8050. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

In some embodiments, the invention provides an isolated antibody or antigen-binding fragment thereof which specifically binds to the same IRAK4 epitope as a reference monoclonal antibody secreted by a hybridoma cell line selected from the group consisting of 101C2, 102C4, 103B6, and 142B2.

In some embodiments, the invention provides an isolated antibody or antigen-binding fragment thereof which specifically binds to IRAK4, where the antibody or fragment competitively inhibits a reference monoclonal antibody secreted by a hybridoma cell line selected from the group consisting of 101C2, 102C4, 103B6, and 142B2.

In some embodiments, the invention provides an isolated antibody or antigen-binding fragment thereof which specifically binds to IRAK4, where the antibody or fragment thereof comprises an antigen binding domain identical to that present in a reference monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the heavy chain variable region (VH) of the antibody or fragment thereof comprises an amino acid sequence at least 80%, 85%, 90% or 95% identical to a reference VH amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the light chain variable region (VL) of the antibody or fragment thereof comprises an amino acid sequence at least 80%, 85%, 90% or 95% identical to a reference VL amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH of the antibody or fragment thereof comprises an amino acid sequence identical, except for 20 or fewer conservative amino acid substitutions, to a reference VH amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VL of the antibody or fragment thereof comprises an amino acid sequence identical, except for 20 or fewer conservative amino acid substitutions, to a reference VL amino acid sequence present in a reference monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH of the antibody or fragment thereof comprises an amino acid sequence present in a reference monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VL of the antibody or fragment thereof comprises an amino acid sequence present in a reference monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH and VL of the antibody or fragment thereof comprise, respectively, amino acid sequences at least at least 80%, 85%, 90% or 95% identical to reference VH and VL amino acid sequences present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH and VL of the antibody or fragment thereof comprise, respectively, amino acid sequences identical, except for 20 or fewer conservative amino acid substitutions each, to reference VH and VL amino acid sequences present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH and VL of the antibody or fragment thereof comprise, respectively, VH and VL amino acid sequences present in a reference monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH of the antibody or fragment thereof comprises a Kabat heavy chain complementarity determining region-1 (VH-CDR1) amino acid sequence identical, except for two or fewer amino acid substitutions, to a reference VH-CDR1 amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH of the antibody or fragment thereof comprises a Kabat heavy chain complementarity determining region-2 (VH-CDR2) amino acid sequence identical, except for four or fewer amino acid substitutions, to a reference VH-CDR2 amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH of the antibody or fragment thereof comprises a Kabat heavy chain complementarity determining region-3 (VH-CDR3) amino acid sequence identical, except for four or fewer amino acid substitutions, to a reference VH-CDR3 amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VL of the antibody or fragment thereof comprises a Kabat light chain complementarity determining region-1 (VL-CDR1) amino acid sequence identical, except for four or fewer amino acid substitutions, to a reference VL-CDR1 amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VL of the antibody or fragment thereof comprises a Kabat light chain complementarity determining region-2 (VL-CDR2) amino acid sequence identical, except for two or fewer amino acid substitutions, to a reference VL-CDR2 amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VL of the antibody or fragment thereof comprises a Kabat light chain complementarity determining region-3 (VL-CDR3) amino acid sequence identical, except for four or fewer amino acid substitutions, to a reference VL-CDR3 amino acid sequence present in a monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH of the antibody or fragment thereof comprises VH-CDR1, VH-CDR2, and VH-CDR3 amino acid sequences present in a reference monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VH of the antibody or fragment thereof comprises VH-CDR1, VH-CDR2, and VH-CDR3 amino acid sequences present in a reference monoclonal antibody secreted by a hybridoma cell line selected from the group consisting of 101C2, 102C4, 103B6, and 142B2.

In some embodiments, the invention provides an isolated antibody or fragment thereof which specifically binds to IRAK4, where the VL of the antibody or fragment thereof comprises VL-CDR1; VL-CDR2, and VL-CDR3 amino acid sequences present in a reference monoclonal antibody secreted by a hybridoma cell line selected from the group consisting of 101C2, 102C4, 103B6, and 142B2, except for one, two, three, or four amino acid substitutions in at least one of said VL-CDRs.

In some embodiments, the invention provides an isolated antibody or fragment thereof which specifically binds to IRAK4, where the VL of the antibody or fragment thereof comprises VL-CDR1, VL-CDR2, and VL-CDR3 amino acid sequences present in a reference monoclonal antibody secreted by a hybridoma cell line selected from the group consisting of 101C2, 102C4, 103B6, and 142B2.

In various embodiments of the above-described antibodies or fragments thereof, the VH framework regions and/or VL framework regions are human, except for five or fewer amino acid substitutions.

In some embodiments, the above-described antibodies or fragments thereof bind to a linear epitope or a non-linear conformation epitope.

In some embodiments, the above-described antibodies or fragments thereof are multivalent, and comprise at least two heavy chains and at least two light chains.

In some embodiments, the above-described antibodies or fragments thereof are multispecific. In further embodiments, the above-described antibodies or fragments thereof are bispecific.

In various embodiments of the above-described antibodies or fragments thereof, the heavy and light chain variable domains are fully human.

In various embodiments of the above-described antibodies or fragments thereof, the heavy and light chain variable domains are murine.

In various embodiments, the above-described antibodies or fragments thereof are humanized.

In various embodiments, the above-described antibodies or fragments thereof are chimeric.

In various embodiments, the above-described antibodies or fragments thereof are primatized.

In various embodiments, the above-described antibodies or fragments thereof are fully human.

In certain embodiments, the above-described antibodies or fragments thereof are Fab fragments, Fab′ fragments, F(ab)₂ fragments, or Fv fragments.

In certain embodiments, the above-described antibodies are single chain antibodies.

In certain embodiments, the above-described antibodies or fragments thereof comprise light chain constant regions selected from the group consisting of a human kappa constant region and a human lambda constant region.

In certain embodiments, the above-described antibodies or fragments thereof comprise a heavy chain constant region or fragment thereof. In further embodiments, the heavy chain constant region or fragment thereof is selected from the group consisting of human IgA (alpha), IgD (delta), IgE (epsilon), IgG (gamma), and IgM (mu).

In some embodiments, the above-described antibodies or fragments thereof specifically bind to an phosphorylated-IRAK4 polypeptide or fragment thereof, or an phosphorylated-IRAK4 variant polypeptide, with an affinity characterized by a dissociation constant (K_{D}) which is less than the K_{D} for said reference monoclonal antibody. In further embodiments, the dissociation constant (K_{D}) is no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments, the above-described antibodies or fragments thereof preferentially bind to a human phosphorylated-IRAK4 polypeptide or fragment thereof, relative to a murine phosphorylated-IRAK4 polypeptide or fragment thereof or a non-human primate phosphorylated-IRAK4 polypeptide or fragment thereof.

In certain other embodiments, the above described antibodies or fragments thereof bind to human phosphorylated-IRAK4 polypeptide or fragment thereof, and also binds to a non-human primate IRAK4 polypeptide or fragment thereof.

In some embodiments, the above described antibodies or fragments thereof are useful in methods of screening compounds to identify those compounds which inhibit or enhance IRAK4 kinase activity.

In further embodiments, the above described antibodies or fragments thereof further comprise a heterologous polypeptide fused thereto.

In some embodiments, the above described antibodies or fragments thereof are conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In further embodiments, the detectable label is selected from the group consisting of an enzyme, a fluorescent label, a chemiluminescent label, a bioluminescent label, a radioactive label, or a combination of two or more of any said detectable labels.

In additional embodiments, the invention includes compositions comprising the above-described antibodies or fragments thereof, and a carrier.

In one embodiment, an IRAK4 antibody, e.g., an antibody of the invention is a bispecific IRAK4 antibody, binding polypeptide, or antibody, e.g., a bispecific antibody, minibody, domain deleted antibody, or fusion protein having binding specificity for more than one epitope, e.g., more than one antigen or more than one epitope on the same antigen. In one embodiment, a bispecific IRAK4 antibody, binding polypeptide, or antibody has at least one binding domain specific for at least one epitope on a target polypeptide disclosed herein, e.g., phosphorylated-IRAK4. In another embodiment, a bispecific IRAK4 antibody, binding polypeptide, or antibody has at least one binding domain specific for an epitope on a target polypeptide and at least one target binding domain specific for a drug or toxin. In yet another embodiment, a bispecific IRAK4 antibody, binding polypeptide, or antibody has at least one binding domain specific for an epitope on a target polypeptide disclosed herein, and at least one binding domain specific for a prodrug. A bispecific IRAK4antibody, binding polypeptide, or antibody may be a tetravalent antibody that has two target binding domains specific for an epitope of a target polypeptide disclosed herein and two target binding domains specific for a second target. Thus, a tetravalent bispecific IRAK4antibody, binding polypeptide, or antibody may be bivalent for each specificity.

IRAK4 antibodies, or antigen-binding fragments, variants, or derivatives thereof of the invention, as known by those of ordinary skill in the art, can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Accordingly, certain embodiments of the invention include an IRAK4 antibody, or antigen-binding fragment, variant, or derivative thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the detection, diagnostic, and/or prognostic methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the C_{H}2 domain will be deleted.

In certain IRAK4 antibodies, or antigen-binding fragments, variants_{;} or derivatives thereof described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of IRAK4 antibodies, or antigen-binding fragments, variants, or derivatives thereof of the invention can be made from whole precursor or parent antibodies using techniques known in the art. Exemplary techniques are discussed in more detail herein.

In certain embodiments both the variable and constant regions of IRAK4 antibodies, or antigen-binding fragments, variants, or derivatives thereof are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human anti bodies can likewise be produced by various display technologies, e.g., phage display or other viral display systems, as described in more detail elsewhere herein.

IRAK4 antibodies, or antigen-binding fragments, variants, or derivatives thereof of the invention can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

IRAK4 antibodies, or antigen-binding fragments, variants, or derivatives thereof of the invention also include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In one embodiment, IRAK4 antibodies, or antigen-binding fragments, variants, or derivatives thereof of the invention are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, V_{H} and V_{L} sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative V_{H} and V_{L} sequences are designed comprising combinations of amino acid substitutions and these sequences are subsequently incorporated into a range of binding polypeptides, e.g., IRAK4-specific antibodies or immunospecific fragments thereof for use in the detection, diagnostic, and/or prognostic methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

IRAK4 antibodies, or antigen-binding fragments, variants, or derivatives thereof of the invention may be generated by any suitable method known in the art. Polyclonal antibodies to an antigen of interest can be produced by various procedures well known in the art. For example, an IRAK4 antibody, e.g., a binding polypeptide, e.g., an IRAK4-specific antibody or immunospecific fragment thereof can be administered to various host animals including, but not limited to, rabbits, mice, rats, chickens, hamsters, goats, donkeys, etc., to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof_{:} For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using IRAK4 knockout mice to increase the regions of epitope recognition. Monoclonal antibodies can be prepared using a wide variety of techniques known in the art including the use of hybridoma and recombinant and phage display technology as described elsewhere herein.

Using art recognized protocols, in one example, antibodies are raised in mammals by multiple subcutaneous or intraperitoneal injections of the relevant antigen (e.g., IRAK4 phosphopeptides, full-length phosphorylated IRAK-4, constitutively activated (i.e., phosphorylated) forms of IRAK4) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and re-growth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Antibody fragments that recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain.

Those skilled in the art will also appreciate that DNA encoding antibodies or antibody fragments (e.g., antigen binding sites) may also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. For example, DNA sequences encoding V_{H} and V_{L} regions are amplified from animal cDNA libraries (e.g., human or murine cDNA libraries of lymphoid tissues) or synthetic cDNA libraries. In certain embodiments, the DNA encoding the V_{H} and V_{L} regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} or V_{L} regions are usually recombinantly fused to either the phage gene III or gene VIII. Phage expressing an antigen binding domain that binds to an antigen of interest (i.e., a phosphorylated-IRAK4 polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from,a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988). See also, U.S. Pat. No. 5,565,332.)

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7(5):437-444 (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In one embodiment, an IRAK4 antibody of the invention comprises at least one heavy or light chain CDR of an antibody molecule. In another embodiment, an IRAK4 antibody of the invention comprises at least two CDRs from one or more antibody molecules. In another embodiment, an IRAK4 antibody of the invention comprises at least three CDRs from one or more antibody molecules. In another embodiment, an IRAK4 antibody of the invention comprises at least four CDRs from one or more antibody molecules. In another embodiment, an IRAK4 antibody of the invention comprises at least five CDRs from one or more antibody molecules. In another embodiment, an IRAK4 antibody of the invention comprises at least six CDRs from one or more antibody molecules. Exemplary antibody molecules comprising at least one CDR that can be included in the subject IRAK4 antibodies are described herein.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., IRAK4. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the V_{H} and V_{L} genes can be amplified using, e.g., RT-PCR. The V_{H} and V_{L} genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies for use in the diagnostic and therapeutic methods disclosed herein can be produced by any method known in the art for the synthesis of antibodies, in particular, by chemical synthesis or preferably, by recombinant expression techniques as described herein.

In one embodiment, an IRAK4 antibody, or antigen-binding fragment, variant, or derivative thereof of the invention comprises a synthetic constant region wherein one or more domains are partially or entirely deleted (“domain-deleted antibodies”). In certain embodiments compatible modified antibodies will comprise domain deleted constructs or variants wherein the entire C_{H}2 domain has been removed (ΔC_{H}2 constructs). For other embodiments a short connecting peptide may be substituted for the deleted domain to provide flexibility and freedom of movement for the variable region. Those skilled in the art will appreciate that such constructs are particularly preferred due to the regulatory properties of the C_{H}2 domain on the catabolic rate of the antibody. Domain deleted constructs can be derived using a vector (e.g., from Biogen IDEC Incorporated) encoding an IgG₁ human constant domain (see, e.g., WO 02/060955A2 and WO02/096948A2). This exemplary vector was engineered to delete the C_{H}2 domain and provide a synthetic vector expressing a domain deleted IgG₁ constant region.

In certain embodiments, IRAK4 antibodies, or antigen-binding fragments, variants, or derivatives thereof of the invention are minibodies. Minibodies can be made using methods described in the art (see, e.g., see e.g., U.S. Pat. No. 5,837,821 or WO 94/09817A1).

In one embodiment, an IRAK4 antibody, or antigen-binding fragment, variant, or derivative thereof of the invention comprises an immunoglobulin heavy chain having deletion or substitution of a few or even a single amino acid as long as it permits association between the monomeric subunits. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides antibodies that comprise, consist essentially of, or consist of, variants (including derivatives) of antibody molecules (e.g., the V_{H} regions and/or V_{L} regions) described herein, which antibodies or fragments thereof immunospecifically bind to an IRAK4 polypeptide or fragment or variant thereof. Standard techniques known to those of skill in the art can be used to introduce mutations in the nucleotide sequence encoding an IRAK4 antibody, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V_{H} region, V_{H}CDR1, V_{H}CDR2, V_{H}CDR3, V_{L} region, V_{L}CDR1, V_{L}CDR2, or V_{L}CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an IRAK4 polypeptide).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one phosphorylated epitope of an IRAK4 polypeptide) can be determined using techniques described herein or by routinely modifying techniques known in the art.

### Polynucleotides Encoding IRAK4 Antibodies

The present invention also provides for nucleic acid molecules encoding IRAK4 antibodies, or antigen-binding fragments, variants, or derivatives thereof of the invention.

The present invention also provides for nucleic acid molecules encoding IRAK4 antibodies, or antigen-binding fragments, variants, or derivatives thereof of the invention.

In certain embodiments, an antibody or antigen-binding fragment thereof comprising, consisting essentially of, or consisting of a VH encoded by one or more of the polynucleotides described above specifically or preferentially binds to the same epitope as a monoclonal antibody secreted by a hybridoma cell line selected from the group consisting of 101C2, 102C4, 103B6, and 142B2, or will competitively inhibit such a monoclonal antibody from binding to phosphorylated-IRAK4.

In certain embodiments, an antibody or antigen-binding fragment thereof comprising, consisting essentially of, or consisting of a VH encoded by one or more of the polynucleotides described above specifically or preferentially binds to a phosphorylated-IRAK4 polypeptide or fragment thereof, or a phosphorylated-IRAK4 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Certain embodiments of the invention include an isolated polynucleotide comprising a nucleic acid which encodes an antibody VH polypeptide, where the amino acid sequence of the VH polypeptide is at least 80%, 85%, 90% or 95% identical to a reference VH polypeptide amino acid sequence present in a monoclonal antibody secreted by a hybridoma cell line selected from the group consisting of 101C2, 102C4, 103B6, and 142B2; and wherein an antibody or antigen binding fragment thereof comprising the VH polypeptide specifically binds to phosphorylated-IRAK4.

In certain embodiments, the nucleotide sequence encoding the VH polypeptide is optimized for increased expression without changing the amino acid sequence of the VH polypeptide. In further embodiments, the optimization comprises identification and removal of splice donor and splice acceptor sites and/or optimization of codon usage for the cells expressing the polynucleotide.

In certain embodiments, an antibody or antigen-binding fragment thereof comprising, consisting essentially of, or consisting of a VL encoded by one or more of the polynucleotides described above specifically or preferentially binds to the same epitope as a monoclonal antibody secreted by a hybridoma cell line selected from the group consisting of 101C2, 102C4, 103B6, and 142B2, or will competitively inhibit such a monoclonal antibody from binding to phosphorylated-IRAK4.

In certain embodiments, an antibody or antigen-binding fragment thereof comprising, consisting essentially of, or consisting of a VL encoded by one or more of the polynucleotides described above specifically or preferentially binds to an IRAK4 polypeptide or fragment thereof, or a IRAK4 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments, the invention provides an isolated polynucleotide comprising a nucleic acid which encodes an antibody VL polypeptide, where the amino acid sequence of the VL polypeptide is at least 80%, 85%, 90% or 95% identical to a reference VL polypeptide amino acid sequence present in a monoclonal antibody secreted by a hybridoma cell line selected from the group consisting of 101C2, 102C4, 103B6, and 142B2; and wherein an antibody or antigen binding fragment thereof comprising the VL polypeptide specifically binds to phosphorylated-IRAK4.

In certain embodiments, the nucleotide sequence encoding the VL polypeptide is optimized for increased expression without changing the amino acid sequence of said VL polypeptide. In further embodiments, the optimization comprises identification and removal of splice donor and splice acceptor sites and/or optimization of codon usage for the cells expressing the polynucleotide.

In certain other embodiments, the invention provides an isolated polynucleotide comprising a nucleic acid which encodes an antibody VH polypeptide, where the amino acid sequence of the VH polypeptide is identical, except for 20 or fewer conservative amino acid substitutions, to a reference VH polypeptide amino acid sequence present in a monoclonal antibody secreted by a hybridoma cell line selected from the group consisting of 101C2, 102C4, 103B6, and 142B2; and wherein an antibody or antigen binding fragment thereof comprising said VH polypeptide specifically binds to phosphorylated-IRAK4.

In some embodiments, the invention provides an isolated polynucleotide comprising a nucleic acid which encodes an antibody VL polypeptide, where the amino acid sequence of the VL polypeptide is identical, except for 20 or fewer conservative amino acid substitutions, to a reference VL polypeptide amino acid sequence present in a monoclonal antibody secreted by a hybridoma cell line selected from the group consisting of 101C2, 102C4, 103B6, and 142B2; and wherein an antibody or antigen binding fragment thereof comprising said VL polypeptide specifically binds to phosphorylated-IRAK4.

In some embodiments, the invention provides an isolated polynucleotide comprising a nucleic acid which encodes a VH-CDR1 amino acid sequence identical, except for two or fewer amino acid substitutions, to a reference VH-CDR1 amino acid sequence present in a monoclonal antibody secreted by a hybridoma cell line selected from the group consisting of 101C2, 102C4, 103B6, and 142B2; and wherein an antibody or antigen binding fragment thereof comprising the VH-CDR1 specifically binds to phosphorylated-IRAK4.

In some embodiments, the invention provides an isolated polynucleotide comprising a nucleic acid which encodes a VH-CDR2 amino acid sequence identical, except for four or fewer amino acid substitutions, to a reference VH-CDR2 amino acid sequence present in a monoclonal antibody secreted by a hybridoma cell line selected from the group consisting of 101C2, 102C4, 103B6, and 142B2; and wherein an antibody or antigen binding fragment thereof comprising the VH-CDR2 specifically binds to phosphorylated-IRAK4.

In some embodiments, the invention provides an isolated polynucleotide comprising a nucleic acid which encodes a VH-CDR3 amino acid sequence identical, except for four or fewer amino acid substitutions, to a reference VH-CDR3 amino acid sequence present' in a monoclonal antibody secreted by a hybridoma cell line selected from the group consisting of 101C2, 102C4, 103B6, and 142B2; and wherein an antibody or antigen binding fragment thereof comprising the VH-CDR3 specifically binds to phosphorylated-IRAK4.

In some embodiments, the invention provides an isolated polynucleotide comprising a nucleic acid which encodes a VL-CDR1 amino acid sequence identical, except for four or fewer amino acid substitutions, to a reference VL-CDR1 amino acid sequence present in a monoclonal antibody secreted by a hybridoma cell line selected from the group consisting of 101C2, 102C4, 103B6, and 142B2; and wherein an antibody or antigen binding fragment thereof comprising the VL-CDR1 specifically binds to phosphorylated-IRAK4.

In some embodiments, the invention provides an isolated polynucleotide comprising a nucleic acid which encodes a VL-CDR2 amino acid sequence identical, except for two or fewer amino acid substitutions, to a reference VL-CDR2 amino acid sequence present in a monoclonal antibody secreted by a hybridoma cell line selected from the group consisting of 101C2, 102C4, 103B6, and 142B2; and wherein an antibody or antigen binding fragment thereof comprising said VL-CDR2 specifically binds to phosphorylated-IRAK4.

In some embodiments, the invention provides an isolated polynucleotide comprising a nucleic acid which encodes a VL-CDR3 amino acid sequence identical, except for four or fewer amino acid substitutions, to a reference VL-CDR3 amino acid sequence present in a monoclonal antibody secreted by a hybridoma cell line selected from the group consisting of 101C2, 102C4, 103B6, and 142B2; and wherein an antibody or antigen binding fragment thereof comprising said VL-CDR3 specifically binds to phosphorylated-IRAK4.

In some embodiments, the invention provides an isolated polynucleotide comprising a nucleic acid which encodes an antibody VH polypeptide, where the VH polypeptide comprises VH-CDR1, VH-CDR2, and VH-CDR3 amino acid sequences present in a monoclonal antibody secreted by a hybridoma cell line selected from the group consisting of 101C2, 102C4, 103B6, and 142B2; and where an antibody or antigen binding fragment thereof comprising the VL-CDR3 specifically binds to phosphorylated-IRAK4.

In some embodiments, the invention provides an isolated polynucleotide comprising a nucleic acid which encodes an antibody VL polypeptide, wherein said VL polypeptide comprises VH-CDR1, VH-CDR2, and VH-CDR3 amino acid sequences present in a monoclonal antibody secreted by a hybridoma cell line selected from the group consisting of 101C2, 102C4, 103B6, and 142B2; and wherein an antibody or antigen binding fragment thereof comprising said VL-CDR3 specifically binds to phosphorylated-IRAK4.

In some embodiments, the above-described polynucleotides further comprise a nucleic acid encoding a signal peptide fused to the antibody VH polypeptide or the antibody VL polypeptide.

In certain other embodiments, the above-described polynucleotides further comprise a nucleic acid encoding a heavy chain constant region CH1 domain fused to the VH polypeptide, encoding a heavy chain constant region CH2 domain fused to the VH polypeptide, encoding a heavy chain constant region CH3 domain fused to the VH polypeptide, or encoding a heavy chain hinge region fused to said VH polypeptide. In further embodiments, the heavy chain constant region is human IgG. In further embodiments, the heavy chain constant region is human IgG4. In certain other embodiments, the IgG is mutagenized to remove glycosylation sites. In further embodiments, the IgG mutations comprise S241P and T318A using the Kabat numbering system.

In some embodiments, the above-described polynucleotides comprise a nucleic acid encoding a light chain constant region domain fused to said VL polypeptide.

In various embodiments of the above-described polynucleotides, the antibody or antigen-binding fragment thereof comprising a polypeptide encoded by the nucleic acid specifically binds the same phosphorylated-IRAK4 epitope as a reference monoclonal antibody secreted by a hybridoma cell line selected from the group consisting of 101C2, 102C4, 103B6, and 142B2.

In various other embodiments of the above-described polynucleotides, the antibody or antigen-binding fragment thereof comprising a polypeptide encoded by the nucleic acid competitively inhibits a reference monoclonal antibody secreted by a hybridoma cell line selected from the group consisting of 101C2, 102C4, 103B6, and 142B2.

In various embodiments of the above-describe polynucleotides, the framework regions of the VH polypeptide or VL polypeptide are human, except for five or fewer amino acid substitutions.

In various embodiments of the above-described polynucleotides, the invention provides an antibody or antigen-binding fragment thereof comprising the polypeptide encoded by the nucleic acid, that binds to a linear epitope or a non-linear conformational epitope.

In various embodiments of the above-described polynucleotides, the antibody or antigen-binding fragment thereof comprising the polypeptide encoded by the nucleic acid is multivalent, and comprises at least two heavy chains and at least two light chains.

In certain embodiments of the above-described polynucleotides, the antibody or antigen-binding fragment thereof comprising the polypeptide encoded by the nucleic acid is multispecific. In further embodiments, the antibody or antigen-binding fragment thereof comprising the polypeptide encoded by the nucleic acid is bispecific.

In various embodiments of the above-described polynucleotides, the antibody or antigen-binding fragment thereof comprising the polypeptide encoded by the nucleic acid comprises heavy and light chain variable domains which are fully human. In further embodiments, the heavy and light chain variable domains are identical to those of a reference monoclonal antibody secreted by a hybridoma cell line selected from the group consisting of 101C2, 102C4, 103B6, and 142B2.

In certain other embodiments of the above-described polynucleotides, the antibody or antigen-binding fragment thereof comprising the polypeptide encoded by the nucleic acid comprises heavy and light chain variable domains which are murine. In further embodiments, the heavy and light chain variable domains are identical to those of a reference monoclonal antibody produced by a hybridoma cell line selected from the group consisting of 101C2, 102C4, 103B6, and 142B2.

In various embodiments of the above-described polynucleotides, the antibody or antigen-binding fragment thereof comprising the polypeptide encoded by the nucleic acid is humanized.

In various embodiments of the above-described polynucleotides, the antibody or antigen-binding fragment thereof comprising the polypeptide encoded by the nucleic acid is primatized.

In various embodiments of the above-described polynucleotides, the antibody or antigen-binding fragment thereof comprising the polypeptide encoded by the nucleic acid is chimeric.

In some embodiments of the above-described polynucleotides, the antibody or antigen-binding fragment thereof comprising the polypeptide encoded by the nucleic acid is fully human.

In various embodiments of the above-described polynucleotides, the antibody or antigen-binding fragment thereof comprising the polypeptide encoded by the nucleic acid is an Fab fragment, an Fab′ fragment, an F(ab)₂ fragment, or an Fv fragment. In certain embodiments of the above-described polynucleotides, the antibody or antigen-binding fragment thereof comprising the polypeptide encoded by the nucleic acid is a single chain antibody.

In some embodiments of the above-described polynucleotides, the antibody or antigen-binding fragment thereof comprising the polypeptide encoded by the nucleic acid specifically binds to an phosphorylated-IRAK4 polypeptide or fragment thereof, or an phosphorylated-IRAK4 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments of the above-described polynucleotides, the antibody or antigen-binding fragment thereof comprising the polypeptide encoded by the nucleic acid preferentially binds to a human phosphorylated-IRAK4 polypeptide or fragment thereof, relative to a murine phosphorylated-IRAK4 polypeptide or fragment thereof or a non-human primate phosphorylated-IRAK4 polypeptide or fragment thereof.

In some embodiments of the above-described polynucleotides, the antibody or antigen-binding fragment thereof comprising the polypeptide encoded by the nucleic acid binds to a human phosphorylated-IRAK4 polypeptide or fragment thereof, and also binds to a non-human primate phosphorylated-IRAK4 polypeptide or fragment thereof.

In some embodiments, the above-described polynucleotides further comprise a nucleic acid encoding a heterologous polypeptide.

In some embodiments of the above-described polynucleotides, the antibody or antigen-binding fragment thereof comprising the polypeptide encoded by the nucleic acid is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In certain other embodiments, the detectable label is selected from the group consisting of an enzyme, a fluorescent label, a chemiluminescent label, a bioluminescent label, a radioactive label, or a combination of two or more of any said detectable labels.

In some embodiments, the invention provides compositions comprising the above-described polynucleotides.

In certain other embodiments, the invention provides vectors comprising the above-described polynucleotides. In further embodiments, the polynucleotides are operably associated with a promoter. In additional embodiments, the invention provides host cells comprising such vectors. In further embodiments, the invention provides vectors where the polynucleotide is operably associated with a promoter.

In additional embodiments, the invention provides a method of producing an antibody or fragment thereof which specifically binds phosphorylated-IRAK4, comprising culturing a host cell containing a vector comprising the above-described polynucleotides, and recovering said antibody, or fragment thereof. In further embodiments, the invention provides an isolated polypeptide produced by the above-described method.

In some embodiments, the invention provides isolated polypeptides encoded by the above-described polynucleotides.

In further embodiments of the above-described polypeptides, the antibody or fragment thereof comprising the polypeptide specifically binds to IGF-1R. Other embodiments include the isolated antibody or fragment thereof comprising the above-described polypeptides.

In some embodiments, the invention provides a composition comprising an isolated VH encoding polynucleotide and an isolated VL encoding polynucleotide, where the VH encoding polynucleotide and the VL encoding polynucleotide, respectively, comprise nucleic acids encoding amino acid sequences at least 80%, 85%, 90% or 95% identical to reference VH and VL amino acid sequences present in a monoclonal antibody produced by a hybridoma cell line selected from the group consisting of 101C2, 102C4, 103B6, and 142B2; and where an antibody or fragment thereof encoded by the VH and VL encoding polynucleotides specifically binds phosphorylated-IRAK4. In further embodiments, the VH encoding polynucleotide and said VL encoding polynucleotide, respectively, comprise nucleic acids encoding VH and VL amino acid sequences present in a reference monoclonal antibody produced by a hybridoma cell line selected from the group consisting of 101C2, 102C4, 103B6, and 142B2.

In certain other embodiments, the invention provides a composition comprising an isolated VH encoding polynucleotide and an isolated VL encoding polynucleotide, where the VH encoding polynucleotide and the VL encoding polynucleotide, respectively, comprise nucleic acids encoding amino acid sequences identical, except for less than 20 conservative amino acid substitutions, to reference VH and VL amino acid sequences present in a monoclonal antibody produced by a hybridoma cell line selected from the group consisting of 101C2, 102C4, 103B6, and 142B2; and where an antibody or fragment thereof encoded by the VH and VL encoding polynucleotides specifically binds phosphorylated-IRAK4. In further embodiments, the VH encoding polynucleotide encodes a VH polypeptide comprising VH-CDR1, VH-CDR2, and VH-CDR3 amino acid sequences present in a reference monoclonal antibody produced by a hybridoma cell line selected from the group consisting of 101C2, 102C4, 103B6, and 142B2; and where an antibody or fragment thereof encoded by the VH and VL encoding polynucleotides specifically binds phosphorylated-IRAK4.

In various embodiments of the above-described compositions, the VH encoding polynucleotide further comprises a nucleic acid encoding a signal peptide fused to the antibody VH polypeptide.

In various embodiments of the above-described compositions, the VL encoding polynucleotide further comprises a nucleic acid encoding a signal peptide fused to the antibody VL polypeptide.

In some embodiments of the above-described compositions, the VH encoding polynucleotide further comprises a nucleic acid encoding a heavy chain constant region CH1 domain fused to the VH polypeptide, further comprises a nucleic acid encoding a heavy chain constant region CH2 domain fused to the VH polypeptide, further comprises a nucleic acid encoding a heavy chain constant region CH3 domain fused to the VH polypeptide, or further comprises a nucleic acid encoding a heavy chain hinge region fused to the VH polypeptide. In further embodiments, the heavy chain constant region is human IgG. In further embodiments, the heavy chain constant region is human IgG4. In certain other embodiments, the IgG is mutagenized to remove glycosylation sites. In further embodiments, the IgG mutations comprise S241P and T318A using the Kabat numbering system.

In some embodiments of the above-described compositions, the VL encoding polynucleotide further comprises a nucleic acid encoding a light chain constant region domain fused to the VL polypeptide.

In some embodiments of the above-described compositions, the antibody or fragment thereof encoded by the VH and VL encoding polynucleotides specifically binds the same IRAK4 epitope as a reference monoclonal antibody produced by a hybridoma cell line selected from the group consisting of 101C2, 102C4, 103B6, and 142B2.

In some embodiments of the above-described compositions, the antibody or fragment thereof encoded by the VH and VL encoding polynucleotides competitively inhibits a reference monoclonal antibody (produced by a hybridoma cell line selected from the group consisting of 101C2, 102C4, 103B6, and 142B2) from binding to phosphorylated-IRAK4.

In some embodiments of the above-described compositions, the framework regions of the VH and VL polypeptides are human, except for five or fewer amino acid substitutions.

In some embodiments of the above-described compositions, the antibody or fragment thereof encoded by the VH and VL encoding polynucleotides binds to a linear epitope or a non-linear conformational epitope.

In some embodiments of the above-described compositions, the antibody or fragment thereof encoded by the VH and VL encoding polynucleotides is multivalent, and comprises at least two heavy chains and at least two light chains.

In some embodiments of the above-described compositions, the antibody or fragment thereof encoded by the VH and VL encoding polynucleotides is multispecific. In further embodiments, the antibody or fragment thereof encoded by the VH and VL encoding polynucleotides is bispecific.

In some embodiments of the above-described compositions, the antibody or fragment thereof encoded by the VH and VL encoding polynucleotides comprises heavy and light chain variable domains which are fully human. In further embodiments, the heavy and light chain variable domains are identical to those present in a reference monoclonal antibody produced by a hybridoma cell line selected from the group consisting of 101C2, 102C4, 103B6, and 142B2.

In some embodiments of the above-described compositions, the antibody or fragment thereof encoded by the VH and VL encoding polynucleotides comprises heavy and light chain variable domains which are murine. In further embodiments, the heavy and light chain variable domains are identical to those present in a reference monoclonal antibody produced by a hybridoma cell line selected from the group consisting of 101C2, 102C4, 103B6, and 142B2.

In various embodiments of the above-described compositions, the antibody or antigen-binding fragment thereof comprising the polypeptide encoded by the nucleic acid is humanized.

In various embodiments of the above-described compositions, the antibody or antigen-binding fragment thereof comprising the polypeptide encoded by the nucleic acid is primatized.

In various embodiments of the above-described compositions, the antibody or antigen-binding fragment thereof comprising the polypeptide encoded by the nucleic acid is chimeric.

In some embodiments of the above-described compositions, the antibody or antigen-binding fragment thereof comprising the polypeptide encoded by the nucleic acid is fully human.

In various embodiments of the above-described compositions, the antibody or antigen-binding fragment thereof comprising the polypeptide encoded by the nucleic acid is an Fab fragment, an Fab′ fragment, an F(ab)₂ fragment, or an Fv fragment. In certain embodiments of the above-described compositions, the antibody or antigen-binding fragment thereof comprising the polypeptide encoded by the nucleic acid is a single chain antibody.

In some embodiments of the above-described compositions, the antibody or antigen-binding fragment thereof comprising the polypeptide encoded by the nucleic acid specifically binds to an phosphorylated-IRAK4 polypeptide or fragment thereof, or an phosphorylated-IRAK4 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵-M.

In some embodiments of the above-described compositions, the antibody or antigen-binding fragment thereof comprising the polypeptide encoded by the nucleic acid preferentially binds to a human phosphorylated-IRAK4 polypeptide or fragment thereof, relative to a murine phosphorylated-IRAK4 polypeptide or fragment thereof or a non-human primate phosphorylated-IRAK4 polypeptide or fragment thereof.

In some embodiments of the above-described compositions, the antibody or antigen-binding fragment thereof comprising the polypeptide encoded by the nucleic acid binds to a human phosphorylated-IRAK4 polypeptide or fragment thereof, and also binds to a non-human primate phosphorylated-IRAK4 polypeptide or fragment thereof.

In some embodiments, the above-described compositions, the VH encoding polynucleotide, the VL encoding polynucleotide, or both the VH and the VL encoding polynucleotides further comprise a nucleic acid encoding a heterologous polypeptide.

In some embodiments of the above-described compositions, the antibody or antigen-binding fragment thereof comprising the polypeptide encoded by the nucleic acid is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In certain other embodiments, the detectable label is selected from the group consisting of an enzyme, a fluorescent label, a chemiluminescent label, a bioluminescent label, a radioactive label, or a combination of two or more of any said detectable labels.

In some embodiments of the above-described compositions, the VH encoding polynucleotide is contained on a first vector and the VL encoding polynucleotide is contained on a second vector. In further embodiments, the VH encoding polynucleotide is operably associated with a first promoter and the VL encoding polynucleotide is operably associated with a second promoter. In certain other embodiments, the first and second promoters are copies of the same promoter. In further embodiments, the first and second promoters non-identical.

In various embodiments of the above-described compositions, the first vector and the second vector are contained in a single host cell.

In certain other embodiments of the above-described compositions, the first vector and the second vector are contained in a separate host cells.

In some embodiments, the invention provides a method of producing an antibody or fragment thereof which specifically binds phosphorylated-IRAK4, comprising culturing the above-described host cells, and recovering the antibody, or fragment thereof.

In other embodiments, the invention provides a method of producing an antibody or fragment thereof which specifically binds phosphorylated-IRAK4, comprising co-culturing separate host cells, and recovering the antibody, or fragment thereof. In further embodiments of the above-described method, the invention provides combining the VH and VL encoding polypeptides, and recovering the antibody, or fragment thereof.

In some embodiments, the invention provides an antibody or fragment thereof which specifically binds IGF-1R, produced by the above-described methods.

In some embodiments, the invention provides compositions, where the VH encoding polynucleotide and the VL encoding polynucleotide are on the same vector, as well as the vectors therein.

In various embodiments of the above described vectors, the VH encoding polynucleotide and the VL encoding polynucleotide are each operably associated with a promoter.

In various embodiments of the above described vectors, the VH encoding polynucleotide and the VL encoding polynucleotide are fused in frame, are co-transcribed from a single promoter operably associated therewith, and are cotranslated into a single chain antibody or antigen-binding fragment thereof.

In various embodiments of the above described vectors, the VH encoding polynucleotide and said VL encoding polynucleotide are co-transcribed from a single promoter operably associated therewith, but are separately translated. In further embodiments, the vectors further comprise an IRES sequence disposed between the VH encoding polynucleotide and the VL encoding polynucleotide. In certain other embodiments, the polynucleotide encoding a VH and the polynucleotide encoding a VL are separately transcribed, each being operably associated with a separate promoter. In further embodiments, the separate promoters are copies of the same promoter or the separate promoters are non-identical.

In some embodiments, the invention provides host cells comprising the above-described vectors.

In other embodiments, the invention provides a method of producing an antibody or fragment thereof which specifically binds phosphorylated-IRAK4, comprising culturing the above-described host cells, and recovering the antibody, or fragment thereof.

In some embodiments, the invention provides an antibody or fragment thereof which specifically binds phosphorylated-IRAK4, produced by the above-described methods.

Any of the polynucleotides described above may further include additional nucleic acids, encoding, e.g., a signal peptide to direct secretion of the encoded polypeptide, antibody constant regions as described herein, or other heterologous polypeptides as described herein.

The present invention also includes fragments of the polynucleotides of the invention, as described elsewhere. Additionally polynucleotides which encode fusion polynucleotides, Fab fragments, and other derivatives, as described herein, are also contemplated by the invention.

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an IRAK4 antibody, or antigen-binding fragment, variant, or derivative thereof may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other IRAK4 antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other IRAK4 antibody. Amplified nucleic acids generated by PCR may then be cloned into replicable cloning vectors using any method well known in the art.

Once the nucleotide sequence and corresponding amino acid sequence of the IRAK4 antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an IRAK4 antibody, or antigen-binding fragment, variant, or derivative thereof can be composed of any polyribonucleotide or polydeoxribonucleotide, which may be unmodified RNA or DNA or modified RNA or DNA. For example, a polynucleotide encoding IRAK4 antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an IRAK4 antibody, or antigen-binding fragment, variant, or derivative thereof can be composed of triple-stranded regions comprising RNA or DNA or both RNA and DNA. A polynucleotide encoding an IRAK4 antibody, or antigen-binding fragment, variant, or derivative thereof may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

### Fusion Proteins and Antibody Conjugates

As discussed in more detail elsewhere herein, IRAK4 antibodies, or antigen-binding fragments, variants, or derivatives thereof of the invention may further be recombinantly fused to a heterologous polypeptide at the N- or C-terminus or chemically conjugated (including covalent and non-covalent conjugations) to polypeptides or other compositions. For example, phosphorylated-IRAK4 specific antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Phosphorylated-IRAK4 antibodies, or antigen-binding fragments, variants, or derivatives thereof of the invention include derivatives that are modified, i.e., by the covalent attachment of any type of molecule to the antibody such that covalent attachment does not prevent the antibody from binding to Phosphorylated-IRAK4. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Phosphorylated-IRAK4 antibodies, or antigen-binding fragments, variants, or derivatives thereof of the invention can be composed of amino acids joined to each other by peptide bonds or modified peptide bonds, i.e., peptide isosteres, and may contain amino acids other than the 20 gene-encoded amino acids. Phosphorylated-IRAK4-specific antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the Phosphorylated-IRAK4-specific antibody, including the peptide backbone, the amino acid side-chains and the amino or carboxyl termini, or on moieties such as carbohydrates. It will be appreciated that the same type of modification may be present in the same or varying degrees at several sites in a given Phosphorylated-IRAK4-specific antibody. Also, a given Phosphorylated-IRAK4-specific antibody may contain many types of modifications. Phosphorylated-IRAK4-specific antibodies may be branched, for example, as a result of ubiquitination, and they may be cyclic, with or without branching. Cyclic, branched, and branched cyclic Phosphorylated-IRAK4-specific antibodies may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising a phosphorylated-IRAK4 antibody, or antigen-binding fragment, variant, or derivative thereof, and a heterologous polypeptide. The heterologous polypeptide to which the antibody is fused may be useful for function or is useful to target phosphorylated-IRAK4 polypeptide expressing cells. In one embodiment, a fusion protein of the invention comprises, consists essentially of, or consists of, a polypeptide having the amino acid sequence of any one or more of the V_{H} regions of an antibody of the invention or the amino acid sequence of any one or more of the V_{L} regions of an antibody of the invention or fragments or variants thereof, and a heterologous polypeptide sequence. In another embodiment, a fusion protein for use in the detection, diagnostic, and/or prognostic methods disclosed herein comprises, consists essentially of, or consists of a polypeptide having the amino acid sequence of any one, two, three of the V_{H} CDRs of an phosphorylated-IRAK4-specific antibody, or fragments, variants, or derivatives thereof, or the amino acid sequence of any one, two, three of the V_{L} CDRs of an phosphorylated-IRAK4-specific antibody, or fragments, variants, or derivatives thereof, and a heterologous polypeptide sequence. In one embodiment, the fusion protein comprises a polypeptide having the amino acid sequence of a V_{H} CDR3 of an phosphorylated-IRAK4-specific antibody of the present invention, or fragment, derivative, or variant thereof, and a heterologous polypeptide sequence, which fusion protein specifically binds to at least one epitope of phosphorylated-lRAK4. In another embodiment, a fusion protein comprises a polypeptide having the amino acid sequence of at least one V_{H} region of an phosphorylated-IRAK4-specific antibody of the invention and the amino acid sequence of at least one V_{L} region of an phosphorylated-IRAK4-specific antibody of the invention or fragments, derivatives or variants thereof, and a heterologous polypeptide sequence. Preferably, the V_{H} and V_{L} regions of the fusion protein correspond to a single source antibody (or scFv or Fab fragment) which specifically binds at least one epitope of phosphorylated-IRAK4. In yet another embodiment, a fusion protein for use in the detection, diagnostic, and/or prognostic methods disclosed herein comprises a polypeptide having the amino acid sequence of any one, two, three or more of the V_{H} CDRs of an phosphorylated-IRAK4-specific antibody and the amino acid sequence of any one, two, three or more of the V_{L} CDRs of an phosphorylated-IRAK4-specific antibody, or fragments or variants thereof, and a heterologous polypeptide sequence. Preferably, two, three, four, five, six, or more of the V_{H}CDR(s) or V_{L}CDR(s) correspond to single source antibody (or scFv or Fab fragment) of the invention. Nucleic acid molecules encoding these fusion proteins are also encompassed by the invention.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

As discussed elsewhere herein, phosphorylated-IRAK4 antibodies, or antigen-binding fragments, variants, or derivatives thereof of the invention may be fused to heterologous polypeptides to increase the in vivo half life of the polypeptides or for use in immunoassays using methods known in the art. For example, in one embodiment, PEG can be conjugated to the phosphorylated-IRAK4 antibodies of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, phosphorylated-IRAK4 antibodies, or antigen-binding fragments, variants, or derivatives thereof of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). The precise site at which the fusion is made may be selected empirically to optimize the secretion or binding characteristics of the fusion protein. DNA encoding the fusion protein is then transfected into a host cell for expression.

Phosphorylated-IRAK4 antibodies of the present invention may be used in non-conjugated form or may be conjugated to at least one of a variety of molecules, e.g., to improve the therapeutic properties of the molecule, to facilitate target detection, or for imaging or therapy of the patient. Phosphorylated-IRAK4 antibodies, or antigen-binding fragments, variants, or derivatives thereof of the invention can be labeled or conjugated either before or after purification, when purification is performed.

In particular, phosphorylated-IRAK4 antibodies, or antigen-binding fragments, variants, or derivatives thereof of the invention may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the phosphorylated-IRAK4 antibodies, or antigen-binding fragments, variants, or derivatives thereof of the invention are prepared in an analogous manner.

The present invention further encompasses phosphorylated-IRAK4 antibodies, or antigen-binding fragments, variants, or derivatives thereof of the invention conjugated to a diagnostic or therapeutic agent. The phosphorylated-IRAK4 antibodies can be used diagnostically to, for example, monitor the development or progression of a neurological disease as part of a clinical testing procedure to, e.g., determine the efficacy of a given treatment and/or prevention regimen. Detection can be facilitated by coupling the phosphorylated-IRAK4 antibody, or antigen-binding fragment, variant, or derivative thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

An phosphorylated-IRAK4 antibody, or antigen-binding fragment, variant, or derivative thereof also can be detectably labeled by coupling it to a chemiluminescent compound. The presence of the chemiluminescent-tagged phosphorylated-IRAK4 antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an phosphorylated-IRAK4 antibody, or antigen-binding fragment, variant, or derivative thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enrymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the phosphorylated-IRAK4 antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the phosphorylated-IRAK4 antibody, or antigen-binding fragment, variant, or derivative thereof, it is possible to detect the antibody through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

An phosphorylated-IRAK4 antibody, or antigen-binding fragment, variant, or derivative thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to an phosphorylated-IRAK4 antibody, or antigen-binding fragment, variant, or derivative thereof are well known, see, e.g., Anion et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

### Expression of Antibody Polypeptides

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Following manipulation of the isolated genetic material to provide phosphorylated-IRAK4 antibodies, or antigen-binding fragments, variants, or derivatives thereof of the invention, the polynucleotides encoding the phosphorylated-IRAK4 antibodies are typically inserted in an expression vector for introduction into host cells that may be used to produce the desired quantity of phosphorylated-IRAK4 antibody.

Recombinant expression of an antibody, or fragment, derivative or analog thereof, e.g., a heavy or light chain of an antibody which binds to a target molecule described herein, e.g., phosphorylated-IRAK4, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes (preferably human) synthetic as discussed above. In one embodiment, this is effected using a proprietary expression vector of Biogen IDEC, Inc., referred to as NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the phosphorylated-IRAK4 antibodies, or antigen-binding fragments, variants, or derivatives thereof of the invention may be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of phosphorylated-IRAK4 antibodies, e.g., binding polypeptides, e.g., phosphorylated-IRAK4-specific antibodies or immunospecific fragments thereof in eukaryotic host cells. Compatible IRES sequences are disclosed in U.S. Pat. No. 6,193,980 which is also incorporated herein. Those skilled in the art will appreciate that such expression systems may be used to effectively produce the full range of phosphorylated-IRAK4 antibodies disclosed in the instant application.

More generally, once the vector or DNA sequence encoding a monomeric subunit of the phosphorylated-IRAK4 antibody has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB11 (Chinese Hamster Ovary lines, DHFR minus), BELA (human cervical carcinoma), CVI (monkey kidney line), COS (a derivative of CVI with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in TK-, HGPRT- or APRT-cells, respectively. Also, anti-metabolite resistance can be used as the basis of selection for the following genes: DHFR, which confers resistance to methotrexate (Wigler et al., Natl. Acad. Sci. USA 77:357 (1980); O'Hare et al., Proc. Natl. Acad. Sci. USA 78:1527 (1981)); GPT, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993);, TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding phosphorylated-IRAK4 antibodies, or antigen-binding fragments, variants, or derivatives thereof of the invention can also be expressed non-mammalian cells such as bacteria or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trp1 lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002 0123057 A1.

### Diagnostics

The invention further provides a diagnostic method useful for diagnosis of conditions in which IRAK4 is hyper- or hypo-phosphorylated (for example, in immune diseases and disorders such as, but not limited, to inflammation, autoimmune disorders, and recurring bacterial infections), which involves measuring the expression level of phosphorylated-IRAK4 protein in tissue or other cells or body fluid from an individual and comparing the measured expression level with a standard phosphorylated-IRAK4 protein level in normal tissue or body fluid, whereby an increase or decrease in the phosphorylated-IRAK4 expression level compared to the standard is indicative of a disorder. For example, antibodies of the invention may be used to detect IRAK4 hypo-phosphorylation in individuals afflicted with recurrent bacterial infections. Conversely, antibodies of the invention may be used to detect IRAK4 hyper-phosphorylation in individuals afflicted with hyperactive immune disorders (e.g., inflammation, autoimmune disorders, sepsis).

Phosphorylated-IRAK4-specific antibodies can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA), immunoprecipitation, or western blotting. Suitable assays are described in more detail elsewhere herein.

By “assaying the expression level of phosphorylated-IRAK4polypeptide” is intended qualitatively or quantitatively measuring or estimating the level of phosphorylated-IRAK4-polypeptide present in a first biological sample either directly (e.g., by determining or estimating absolute phosphorylated-IRAK4-protein level) or relatively (e.g., by comparing a first biological (“test”) sample to second a second biological control (“normal”) sample without disease or disorder). Preferably, phosphorylated-IRAK4 polypeptide expression level in the first biological sample is measured or estimated and compared to a standard phosphorylated-IRAK4 polypeptide level, the standard being taken from a second biological sample obtained from an individual not having the disorder or being determined by averaging levels from a population of individuals not having the disorder. As will be appreciated in the art, once the “standard” phosphorylated-IRAK4 polypeptide level is known, it can be used repeatedly as a standard for comparison.

By “biological sample” is intended any biological sample obtained from an individual, cell line, tissue culture, or other source of cells potentially containing phosphorylated-IRAK4. Methods for obtaining tissue biopsies and body fluids from mammals are well known in the art.

Phosphorylated-IRAK4 antibodies for use in the diagnostic methods described above include any hosphorylated-IRAK4 antibody which specifically binds to an phosphorylated-IRAK4 gene product, as described elsewhere herein.

### Immunoassays

Phosphorylated-IRAK4 antibodies, or antigen-binding fragments, variants, or derivatives thereof of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4.degree. C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32p or 1251) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

Phosphorylated-IRAK4 antibodies, or antigen-binding fragments, variants, or derivatives thereof of the invention, additionally, be employed histologically, as in immunofluoresence, immunoelectron microscopy or non-immunological assays, for in situ detection of cancer antigen gene products or conserved variants or peptide fragments thereof. In situ detection may be accomplished by removing a histological specimen from a patient, and applying thereto a labeled phosphorylated-IRAK4 antibody, or antigen-binding fragment, variant, or derivative thereof, preferably applied by overlaying the labeled antibody (or fragment) onto a biological sample. Through the use of such a procedure, it is possible to determine not only the presence of phosphorylated-IRAK4 protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for phosphorylated-IRAK4 gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to phosphorylated-IRAK4 or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled phosphorylated-IRAK4 antibody, or antigen-binding fragment, variant, or derivative thereof. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of phosphorylated-IRAK4 antibody, or antigen-binding fragment, variant, or derivative thereof may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIA applications Handbook, version AB (reprinted 1998), BIACORE code No. BR-1001-86; BIAtechnology Handbook, version AB (reprinted 1998), BIACORE code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a monoclonal antibody. Epitope mapping with BIAcore, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIAcore are straightforward to carry out.

For example, one can use a capture molecule to bind the first Mab (monoclonal antibody), followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No: 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses; Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunnology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All publications and patent applications cited throughout this specification are herein incorporated by reference in their entireties to the same extent as if each individual publication or patent application was specifically and individually indicated to be incorporated by reference.

### Examples

### Example A

### Production, Purification, and Auto-/Trans-Phosphorylation of IRAK4 Kinase Domain

Construction of the wild type IRAK4 kinase domain. The wild type IRAK4 kinase domain (Thr-163 to Ser-460 of the full-length protein) was obtained by PCR amplification from a full-length IRAK4 template. The PCR products were digested with restriction enzymes NcoI and EcoRI and cloned directionally into NcoI/EcoRI-digested pFastBac HTA vector (INVITROGEN™, Carlsbad, Calif.) to produce a construct in which the sequence encoding the IRAK4 kinase domain is downstream of sequences encoding a hexahistidine tag (his-tag) and a TEV protease cleavage site. Standard protocols routinely practiced by those of ordinary skill in the art were used to transfect Sf-9 insect cells and to generate and amplify baculovirus stocks.

Construction of Wild Type and T342A, T345A, S346A, T342A/T345A, T342A/S346A, T345A/S346A, and K213A/K214A Mutants of Full-Length IRAK4.

A mammalian cell expression construct of wild-type full length human IRAK 4 (CH402) was obtained by routine PCR amplification methods. In particular, the coding region of human IRAK 4 was PCR amplified from a full length clone using oligonucleotide primer IR1-030:

(SEQ ID NO: 7)
ATAAGAATGCGGCCGCCATGAACAAACCCATAACACCATCAAC
and oligonucleotide primer 1R1-075:
(SEQ ID NO: 8)
ATAGTTTAGCGGCCGCTTAAGAAGCTGTCATCTCTTGC.

PCR amplification was performed with Herculase Hotstart DNA polymerase and reagents and protocol from Stratagene (Cat #600312). The PCR product was digested with Not1, gel purified, and cloned into a mammalian cell expression vector (pV90) in which expression is driven by the CMV immediate early promoter. Additionally, the pV90 vector (thus also, the CH402 construct) contains the human growth hormone 3′ untranslated region and sv2 derived DHFR for selection and amplification.

Vectors encoding IRAK4 kinase domain mutants were constructed using routine site-directed mutagenesis procedures. In particular, all site directed mutagenesis reactions utilized reagents and protocol supplied with Quick Change XL (Stratagene cat #200516). Templates and oligonucleotides used for construction of each mutant are indicated below:

IRAK 4 T342A mutant (CH424)
Wild type IRAK 4 (CH402) with
(SEQ ID NO: 9)
GAGAAGTTTGCCCAGGCAGTCATGACTAGCAGA
and
(SEQ ID NO: 10)
TCTGCTAGTCATGACTGCCTGGGCAAACTTCTC
IR1-152/153)
IRAK 4 T345A mutant (CH428)
Wild type IRAK 4 (CH402) with
(SEQ ID NO: 11)
CCAGACAGTCATGGCTAGCAGAATTGTGG
and
(SEQ ID NO: 12)
CCACAATTCTGCTAGCCATGACTGTCTGG
(IR1-046/047)
IRAK 4 S346A mutant (CH425)
Wild type IRAK 4 (CH402) with
(SEQ ID NO: 13)
GCCCAGACAGTCATGACTGCAAGAATTGTGGGAACAACAGC
and
(SEQ ID NO: 14)
GCTGTTGTTCCCACAATTCTTGCAGTCATGACTGTCTGGGC
(IR1-154/155)
IRAK 4 T342A/T345A double mutant (CH517)
IRAK 4 T342A mutant (CH424) with
(SEQ ID NO: 15)
CCAGGCAGTCATGGCTAGCAGAATTGTG
and
(SEQ ID NO: 16)
CACAATTCTGCTAGCCATGACTGCCTGG
(IR1 -297/298)
IRAK 4 T342A/S346A double mutant (CH628)
IRAK 4 S346A mutant (CH425) with
(SEQ ID NO: 17)
CTTCTGAGAAGTTTGCCCAGGCCGTCATGACTGCAAGAATTG
and
(SEQ ID NO: 18)
CAATTCTTGCAGTCATGACGGCCTGGGCAAACTTCTCAGAAG
(IR1-313/314)
IRAK 4 T345A/S346A double mutant (CH629)
IRAK 4 S346A mutant (CH425) and
(SEQ ID NO: 19)
GCCCAGACAGTCATGGCCGCAAGAATTGTGGG
and
(SEQ ID NO: 20)
CCCACAATTCTTGCGGCCATGACTGTCTGGGC
(IR1-315/316)

IRAK 4 K213A/K214A Double Mutant (CH406)

Full length IRAK 4 in vector pDONR221 (Invitrogen) (CH384) with GTGGCAGTGGCGGCGCTTGCAGC (SEQ ID NO:21) and GCTGCAAGCGCCGCCACTGCCAC (IR1-044/045) (SEQ ID NO:22). A sequence confirmed clone (GJ595) with the KK/AA mutation was used as template for PCR with primers IR1-030/IR1-075 and cloned into mammalian cell expression vector and DNA sequence confirmed as stated for construct 1.

Expression and purification of the wild type IRAK4 kinase domain. The wild type IRAK4 kinase domain was expressed in Sf-9 insect cells (INVITROGEN™ Inc., Carlsbad, Calif.). Cells were grown in a 20 L bioreactor controlled for oxygen at 28° C. in Sf-900 II serum-free medium (Invitrogen, Corp., Carlsbad, Calif.) to a density of ˜2×10⁶ cells/mL. The cells were then infected with baculovirus expressing the IRAK4 kinase domain and grown for 48 hours post-infection. Cells were harvested by centrifugation and stored at −70° C. For purification, all steps were carried out at 4° C. unless stated otherwise. The protein was typically purified from ˜1 kg (wet weight) of cells. 1.1 kg of frozen Sf-9 cells was thawed from −70° C., and resuspended in 5 L of 25 mM Tris-HCl pH 7.5, 2 mM DTT containing the following protease inhibitors: 1 mM benzamidine, 10 μM bestatin, 10 μM E-64, 10 μg/mL leupeptin, 10 μg/mL aprotinin, 5 μM pepstatin A, and 1 mM PMSF (all subsequent references to this mixture will be referred to simply as protease inhibitors). The cells were disrupted by a single pass through a Gaulin APV model 15-15MR-8TBA homogenizer (Wilmington, Mass.) at 5000 psi, and the resulting homogenate centrifuged for 1 h at 22,000 g. The supernatant was decanted and loaded at 100 mL/min (76.4 cm/h) onto a 2 L column (10 cm internal diameter×25 cm) of Q-SEPHAROSE FAST FLOW™ resin (GE Healthcare, Piscatawy, N.J.) that had been equilibrated with 25 mM Tris-HCl pH 7.5, 2 mM DTT, containing protease inhibitors. Once loaded, the column was washed with 10 column volumes (CV) of 25 mM Tris-HCl pH 7.5, 2 mM DTT, containing protease inhibitors, then bound protein eluted at 50 mL/min (38.2 cm/h) with 6 CV of 25 mM Tris-HCl pH 7.5, 2 mM DTT, 0.3 M NaCl, containing protease inhibitors. 2.09 L of 5 M NaCl and 287 mL of 1 M imidazole pH 8.0 (both containing protease inhibitors), were added to bring the NaCl and imidazole concentrations to 1 M and 20 mM, respectively. The diluted sample was then loaded at 20 mL/min (61.1 cm/h) onto a 294 mL column (5 cm internal diameter×15 cm) of NTA Ni²⁺-Superflow resin (QIAGEN®, Inc., Valencia, Calif.) that had been equilibrated with 25 mM Tris-HCl pH 8.0, 1 M NaCl, 20 mM imidazole, 0.5 mM DTT, containing protease inhibitors. Once loaded, the column was washed with 10 CV of 25 mM Tris-HCl pH 8.0, 1 M NaCl, 20 mM imidazole, 0.5 mM DTT, containing protease inhibitors, prior to elution of bound protein at 10 mL/min (30.6 cm/h) with 25 mM Tris-HCl pH 8.0, 1 M NaCl, 200 mM imidazole, 0.5 mM DTT, containing protease inhibitors. 40 mL fractions were collected and analyzed by reducing SDS-PAGE. Fractions containing the histidine-tagged protein were pooled (320 mL), and sufficient 1 M DTT added to bring the concentration to 2 mM. The pool was then concentrated to ˜50 mL with an AMICON® Stirred Cell fitted with a 10,000 molecular weight cut off membrane (Millipore Corp., Bedford, Mass.). The pool was concentrated further to 17 mL with AMICON® CENTRIPREP™ 10 concentrators, then centrifuged for 20 min at 14,500 g to precipitate insoluble material. The supernatant was decanted, and the monomeric protein separated from aggregated material using size exclusion chromatography. Approximately 8 mL samples of the supernatant were loaded (at ambient temperature) at 1.5 mL/min (4.6 cm/h) onto a 1.8 L column (5 cm internal diameter×92 cm) of SUPEROSE™ 12 resin (GE Healthcare Co., Piscataway, N.J.) using 25 mM Tris-HCl pH 7.5, 150 mM NaCl, 0.5 mM DTT as the mobile phase. 6 mL fractions were collected and analyzed by reducing SDS-PAGE. Fractions containing monomeric protein were pooled, mixed with his-tagged TEV protease (INVITROGEN™ Inc.) (1 unit per 8 μg of IRAK4) and incubated at 4° C. for ˜18 h to cleave the tag. A Gly-Ala-Met-Gly sequence, derived from the pFASTBAC™ HTA vector (INVITROGEN™ Inc.), was appended to the N-terminus following cleavage i.e. Thr-163 of IRAK4 was the 5^{th} N-terminal residue. To separate the detagged IRAK4 from the liberated tag, any uncleaved protein, and the TEV protease, the digested sample was loaded under gravity onto a 10 cm (1 cm internal diameter×12.7 cm) column of NTA Ni²⁺-Superflow resin that had been equilibrated with 25 mM Tris-HCl pH 8.0, 1 M NaCl, 20 mM imidazole, 0.5 mM DTT. Once loaded, the column was washed with 4×1 CV washes of 25 mM Tris-HCl pH 8.0, 1 M NaCl, 20 mM imidazole, 0.5 mM DTT, and the flow through and wash fractions pooled. The sample was dialyzed extensively against 25 mM Tris-HCl pH 7.6, 250 mM NaCl, 0.1 mM EDTA, 0.25 mM TCEP. The dialyzed, de-tagged, IRAK4 domain was then aliquoted, flash-frozen with liquid N₂, and stored at −70° C. ˜100 mg of protein was purified from 1 kg of cells.

Determination of Protein Concentration. The concentration of the purified wild type histidine-tagged IRAK4 kinase domain was determined from absorbance measurements at 280 nm using a calculated extinction coefficient of 22,330 L mol⁻¹ cm⁻¹, while an extinction coefficient of 17,210 L mol⁻¹ cm⁻¹ was used following removal of the his-tag (See, Gill, S. C. and von Hippel, P. H. (1989) Anal. Biochem., 182, 319-326).

Auto/transphosphorylation of the wild type IRAK4 kinase domain. 100 μL samples of wild type IRAK4 kinase domain (0.5 mg/mL, 14.8 μM) in a buffer of 50 mM HEPES pH 7.5, 60 mM NaCl, 2 mM DTT, 10 mM MgCl₂, were incubated for 6 h at ambient temperature in the absence or presence of 10 mM ATP. At t=0, 0.5, 1, 2, 4, and 6 h following the addition of ATP (or buffer where ATP was omitted), 90 μL (45 μg, 1.3 nmol) samples were removed and the phosphorylation reaction quenched by the addition of EDTA to 50 mM (final concentration). The samples were immediately frozen on dry ice and stored at −70° C. until analyzed by mass spectrometry as described below.

### Example B

### Generation of Polyclonal Antibodies that Specifically Bind Phosphorylated-IRAK4

Polyclonal antibodies that specifically bind phosphorylated-IRAK4 were generated using rabbits immunized with phosphorylated IRAK4 peptide antigen comprised of the polypeptide sequence:

(SEQ ID NO:3)
Cys-Lys-Phe-Ala-Gln-Thr-Val-Met-Phos-Thr-Ser-
Arg-Ile-Val-Gly-Thr-Thr
(SEQ ID NO:3)
(or, CKFAQTVM^{Phos-}TSRIVGTT )

where “^{Phos-}Thr”/“^{Phos-}T” signifies a phosphorylated Threonine residue, particularly corresponding to phospho-Thr-345 in IRAK4. (Note: The first Cys residue is not native to IRAK4 and was introduced to facilitate conjugation of the peptide to Keyhole Limpet Hemocyanin (KLH) carrier protein.).

Rabbits were injected three times, at three week intervals, with approximately 250 micrograms of peptide antigen prior to a first bleed. Seven to ten days prior to a second bleed another immunization was performed. Overall, rabbits were immunized seven times to obtain sufficiently high titers of anti-phospho-Thr345-IRAK4 antibodies (e.g., useful in ELISA at dilutions >1:125,000).

Antibodies specific for phospho-Thr345-IRAK4 peptide were then purified from the rabbit antiserum. First, antiserum was negatively depleted of antibodies that bind the non-phosphorylated form of the IRAK4 peptide by passing antiserum through a matrix of Thiosepharose 6B conjugated to unphosphorylated IRAK4 peptide. Second, anti-phospho-Thr345-IRAK4 antibodies were purified by affinity purification by passing the eluate through a matrix of Thiosepharose 6B conjugated to phospho-Thr345-IRAK4 peptide. Antibodies were then concentrated using an AMICON concentrator.

The specificity of affinity purified phospho-Thr345-IRAK4 antibodies for phosphorylated IRAK4 versus non-phosphorylated IRAK4 was first verified by ELISA with phosphorylated versus non-phosphorylated peptides conjugated to BSA as the target antigens. Specificity for phosphoThr345 IRAK4 was further demonstrated by immunoblot comparison of IRAK4 kinase domain incubated for 10 minutes in kinase buffer in the presence of ATP and Mg²⁺ (to generate phosphorylated-IRAK4) versus IRAK4 kinase domain incubated in the absence of ATP and Mg²⁺(non-phosphorylated IRAK4). Additionally, the specificity of antibodies for phosphorylated IRAK4 was also demonstrated by immunoblot comparison of lysates from HeLa cells transfected with DNA encoding wild-type full-length IRAK4 (i.e., phosphorylated IRAK4) versus a kinase-inactive IRAK4 mutant (K212A/K213A double mutant; i.e., non-phosphorylated IRAK4):

### Example C

### Generation of Monoclonal Antibodies that Specifically Bind Phosphorylated-IRAK4

Methods for producing monoclonal antibodies are routinely practiced and well known in the art, as exemplified by numerous publications and laboratory manuals describing such procedures. See, for example, Kohler G, and C. Milstein, Continuous cultures of fused cells secreting antibody of predefined specificity, Nature 256: 495-497 (1975); Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981); Howard et al., Basic Methods in Antibody Production and Characterization, CRC Press LLC, Boca Raton, Fla. (2001); and, J. D. Pound, Immunochemical Protocols, Methods in Molecular Biology, Vol. 80, 2^{nd} Ed., Humana Press, Inc. Totowa, N.J. (1998).

Monoclonal antibodies that specifically bind phosphorylated IRAK4 were developed as described below. First, Balb/c mice were immunized with phosphorylated IRAK4 peptide antigen comprised of the polypeptide sequence:

(SEQ ID NO: 3)
Cys-Lys-Phe-Ala-Gln-Thr-Val-Met-Phos-Thr-Ser-
Arg-Ile-Val-Gly-Thr-Thr
(SEQ ID NO:3)

(or, CKFAQTVMPhos-TSRIVGTT)

where “^{Phos-}Thr”/“^{Phos-}T” signifies a phosphorylated Threonine residue, particularly corresponding to phospho-Thr-345 in IRAK4. (Note: The first Cys residue is not native to IRAK4 and was introduced to facilitate conjugation of the peptide to Keyhole Limpet Hemocyanin (KLH) carrier protein.).

Mice were injected subcutaneously with 50-100 micrograms peptide antigen per immunization. Initial immunizations were performed with peptide in Complete Freund's Adjuvant (CFA). Subsequent immunizations were performed using Incomplete Freund's Adjuvant (IFA). Three rounds of immunization were performed at three week intervals. One week after the third immunization, antibody titers were determined by ELISA. Immunizations were repeated in mice exhibiting low serum titers.

After serum titers reached levels sufficient for use in ELISA (at dilutions of greater than 1:100,000), the mouse with the highest serum titer was given a final intraperitoneal (IP) boost of peptide antigen (without adjuvant). Four days after the final boost, the spleen was removed and spleen cells were fused with SP2/0-Ag14 myeloma cells (ATCC Deposit CRL-1581; developed by M. Shulman, C. Wilde, and G. Kohler; submitted by G. Kohler; see also, J. hrununol. 126:317-321 (1981)).

Fused cells were selected by culturing in hybridoma selection medium containing hypoxanthine/aminopterin/thymidine (HAT) at 37° C., 5% CO₂. Seven to twenty-one days after fusion, viable cells were screened by ELISA to identify hybridomas producing antibodies that specifically bind phosphorylated IRAK4 peptide antigen (CKFAQTVM^{Phos-}TSRIVGTT (SEQ ID NO:3)) conjugated to Bovine Serum Albumin (BSA), but not the corresponding unphosphorylated peptide (CKFAQTVMTSRIVGTT (SEQ ID NO:23)) conjugated to BSA. Hybridomas producing antibodies that specifically bound the phosphorylated IRAK4 peptide antigen were selected for further expansion.

Parental clones were isolated and expanded for subsequent harvesting of tissue culture supernatants. Supernatants are used for ELISA and Western blot (immunoblot) analyses. In addition to peptide-based ELISA, the specificity of antibodies for phosphorylated IRAK4 versus non-phosphorylated IRAK4 was further demonstrated by immunoblot comparison of lysates from HeLa cells transfected with DNA encoding wild-type full-length IRAK4 (i.e., phosphorylated IRAK4) versus a kinase-inactive IRAK4 mutant (K212A/K213A double mutant; i.e., non-phosphorylated IRAK4).

Hybridomas producing antibodies with the highest antigen specificity were expanded and cryopreserved. One such hybridoma cell line, IRAK4-101C2 (also referred to herein as “101C2”), was deposited with the American Type Culture Collection (ATCC) on Nov. 30, 2006, and given ATCC Patent Deposit Designation PTA-8050.

### Example D

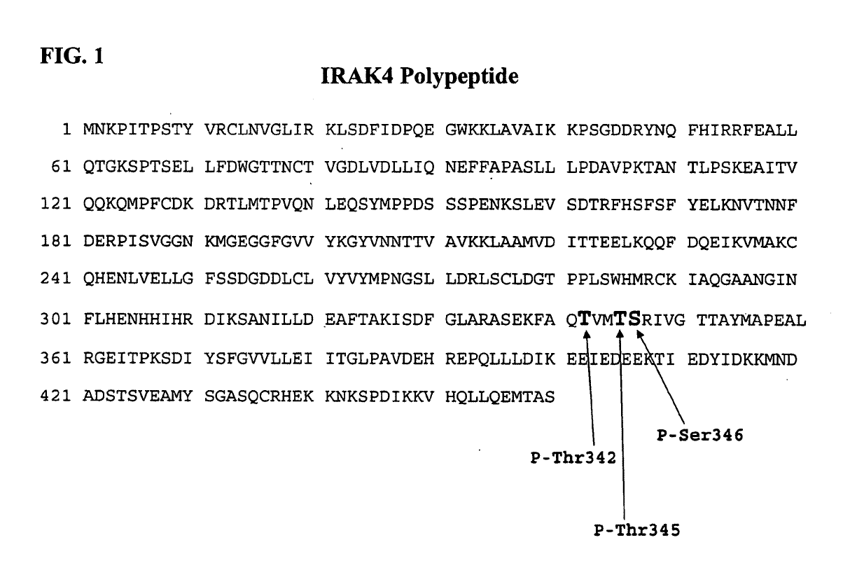

### Mass Spectrometry and Peptide Mapping: IRAK4 is Phosphorylated at Amino Acid Residues Thr342, Thr345, and Ser346 (FIG. 1).

Mass Spectrometry and Peptide Mapping. The mass of the purified wild type IRAK4 kinase domain was determined by electrospray-ionization mass spectrometry (ESI-MS) using a ZMD 4000 single quadrupole mass spectrometer equipped with an electrospray ion source (Waters Corp, Milford, Mass.). Samples were desalted using an on-line HPLC system (2695 Separation Module, Waters Corp., Milford, Mass.) fitted with a Vydac C₄ cartridge (2 mm×5 mm) (Vydac, Columbia, Md.) at 30° C. A 20 minute linear gradient of 5-80% acetonitrile gradient in 0.03% trifluoracetic acid was used for desalting. The flow rate was 0.1 mL/min. The mass spectrometer was operated in the positive ion mode scanning from m/z 600 to 2500 every 6.75 seconds (s). The capilliary voltage, cone voltage, source block temperature, desolvation temperature, and desolvation gas flow were 3000 V, 25 V, 110° C., 230° C., and 10 L/min, respectively. The molecular masses were generated by deconvolution using MaxEnt 1 software (Waters Corp, Milford, Mass.).

Peptide mapping was used to determine the location and site occupancy of phosphate groups attached to the wild type kinase domain following incubation with Mg²⁺/ATP. Protein (14 μg) was denatured and reduced by incubating in 0.1 M Tris-HCl pH 7.6, 6.0 M guanidine hydrochloride, 40 mM DTT, 5 mM EDTA for 4 h at 37° C., then alkylated in the dark at ambient temperature for 30-45 min following the addition iodoacetamide (to 125 mM). The alkylated protein was then recovered by precipitation with 40 volumes of −20° C. ethanol (Pepinsky, R. B., Selective precipitation of proteins from guanidine hydrochloride-containing solutions with ethanol, Anal. Biochem., 195, 177-185 (1991). The solution was stored at −20° C. for 1 h, then centrifuged at 14,000 g for 12 min at 4° C. The supernatant was removed, and the precipitate (˜7 μg) was washed once with −20° C. ethanol. The reduced and alkylated protein was dissolved in 100 μL of 50% acetonitrile, 0.1% trifluroacetic acid (TFA), and dried under vacuum. The protein was resuspended in 50 μL of 0.2 M Tris-HCl pH 8.0, 1 M urea, 10 mM methylamine, 1 mM CaCl₂, containing either 5% (w/w) trypsin or endo Lys C, and incubated at ambient temperature for 9 h (trypsin) or 20 h (endo Lys C). 100 pmole samples of the digested protein were loaded onto a Vydac C₁₈ (218TP51) reverse phase column fitted to a 2690 Alliance Separations Module reverse-phase HPLC (Waters Corp., Milford, Mass.). Peptides were eluted with the following gradient at 70 μL/min where A=0.03% (v/v) TFA and B=acetonitrile, 0.024% (v/v) TFA: 0-60% B in 90 mM, 60-70% B in 10 min, 70% B for 5 min. Eluting peptide were detected on line with a 2487 dual wavelength UV detector and an LCT mass spectrometer (Waters Corp.).

Results: Prior to peptide mapping analysis, a time course experiment was carried out to determine the number of site(s) modified and at what time, following the addition of ATP, was maximal phosphorylation observed. The protein was incubated for 0-6 hours at ambient temperature in the presence of 10 mM Mg2+/ATP. At t=0, 0.5, 1, 2, 4, and 6 hours post ATP addition, samples were removed, the phosphorylation reaction quenched with EDTA, and the samples frozen for mass spectrometry. A control sample was incubated in the presence of 10 mM MgCl₂ but in the absence of ATP, and analyzed at t=0 and 6 hours. For the control sample, the mass of the protein at t=0 and t=6 hours was 33,691 daltons (Da) and 33,690 Da, respectively, indicating that there was no protein modification in the absence of ATP. For the protein incubated in the presence of ATP, no unmodified protein was detected at any of the time points following addition of ATP. Rather, two prominent species with masses of 33,931 Da and 34,010 Da were observed, consistent with IRAK4 kinase domain with 3 and 4 phosphate groups attached (data not shown). The tri- and tetra-phosphorylated species were the major forms detected at all time points. In addition, species with masses consistent with 5 and 6 phosphates attached were detected at all time points, and a mass consistent with 7 phosphates attached was detected at and subsequent to the 2 hour time point. However, the penta-, hexa-, and hepta-phosphorylated forms constituted only a minor percentage of the total population (data not shown).

To determine the sites of phosphorylation and the percentage site occupancy, the sample of IRAK4 kinase domain incubated for 1 hour in the presence of ATP was subject to peptide mapping. As a control, the t=0 sample incubated in the presence of 10 mM MgCl₂ but in the absence of ATP was also subject to peptide mapping. Liquid chromatography-mass spectrometry (LC-MS) analysis of trypsin digests of the control sample identified all of the serine- and threonine-containing peptides directly, except for a short peptide (Ala-335 to Lys-338), containing Ser-336, which was identified by endo LysC peptide mapping. Comparison of the peptide maps derived from the unmodified and phosphorylated IRAK4 kinase domains showed that the tryptic peptide corresponding to residues Phe-339 to Arg-347 was absent in the map of the modified protein. However, a peptide corresponding to Phe-339 to Arg-347 with three phosphate groups attached was detected (data not shown). Since this peptide (Phe-Ala-Gln-Thr-Val-Met-Thr-Ser-Arg) does not contain any potential phosphorylation sites other than the two threonines and the single serine, the data indicated that Thr-342, Thr-345, and Ser-346, all of which are within the activation loop of the kinase domain, were the targets for auto/transphosphorylation. Moreover, endo LysC peptide mapping showed that >95% of the modified protein was phosphorylated on all three of these residues. In addition to the full occupancy of Thr-342, Thr-345, and Ser-346, endo LysC peptide mapping showed that 30% of the protein was phosphorylated on Ser-167 and/or Ser-169. Overall, the peptide mapping data was consistent with the intact mass analysis above, with the exception that the site(s) that constituted the 6th and 7th sites modified were not identified. However, this was not surprising since the level of these forms was very low (data not shown).

### Example E

### Phospho-Thr345-IRAK4 Antibodies Bind Auto-/Trans-Phosphorylated IRAK4

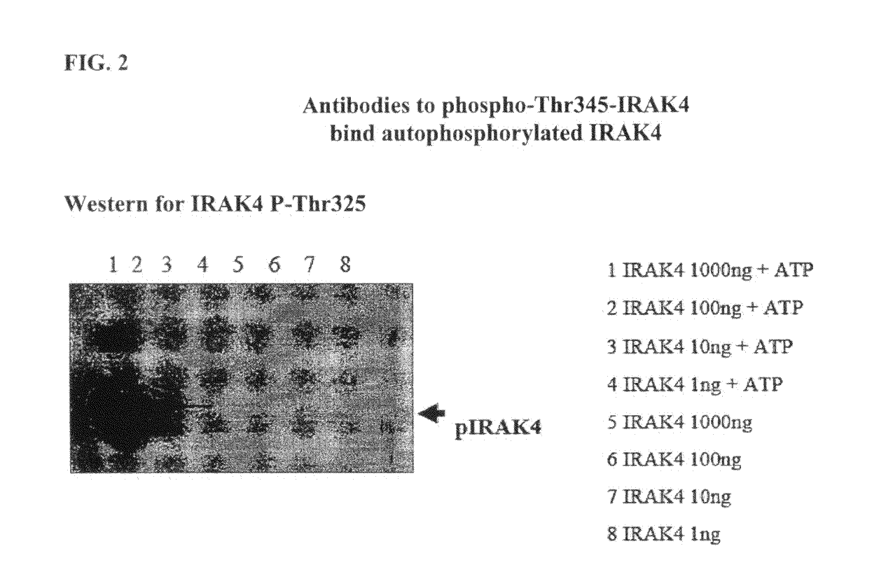

Immunoblot analysis: Western blot (immunoblot) analysis demonstrated the ability of affinity purified polyclonal antibodies to specifically bind to phosphorylated IRAK4 versus unphosphorylated IRAK4. Phosphorylated and unphosphorylated forms of IRAK4 protein (incubated in kinase buffer in the presence (phosphorylated) or absence (unphosphorylated) of ATP and Mg²⁺) were obtained and diluted in 20 mM Tris pH7.5/150 mM NaCl (TBS). Samples containing 1 ng, 10 ng, 100 ng, and 1000 ng were fractionated on a 4-20% tris-glycine minigel under reducing conditions. Fractionated proteins were transferred to a polyvinylidene fluoride membrane (PVDF), blocked for 1 hour in TBS/0.1% Tween20/5% milk (blocking buffer) at room temperature, followed by incubation with anti-IRAK4 phospho-Thr345 polyclonal antibody diluted (2.5 micrograms/mL) in blocking buffer overnight at 4° C. The PVDF membrane (immunoblot) was washed at room temperature with TBST, followed by incubations for 1 hour at room temperature with secondary antibody (donkey-anti-rabbit IgG, horseradish peroxidase linked F(ab′)2 fragment (Amersham Biosciences)) diluted 1:5000 in blocking buffer. Proteins were visualized using AMERSHAM ECL PLUS™ Western Blotting Detection Reagents (GE Healthcare Bio-Sciences Corp., Piscataway, N.J.). See, FIG. 2.

### Example F

### Detection of Phosphorylated IRAK4 with Phospho-Thr345-IRAK4 Antibodies in Cells Transfected with Vector Encoding Wild-Type IRAK4

HeLa cells were grown at 37° C. with 5% CO₂ in MEM Earles (Minimum Essential Media Earles) (INVITROGEN™) with 10% fetal calf serum, 2 mM glutamine, 1 mM sodium pyruvate, 0.1 mM non-essential amino acids, and penicillin/streptomycin (100 ug/mL). For transfection, cells were seeded in a 6-well dish at 7.5×10⁵ cells per well, and transfected the next day using LIPOFECTAMINE™ 2000 (INVITROGEN™). HeLa cells were transfected with various expression plasmids to assess detection of phosphorylated IRAK4 using affinity purified, polyclonal Phospho-Thr345-IRAK4 antibodies.

Expression plasmids containing vector only (control) or encoding IRAK1, IRAK4-K212A (kinase inactive IRAK4 having a Lys-212 to Ala substitution), wild-type IRAK4 (WT), or IRAK4-T345A (IRAK4 with a Thr-345 to Ala substitution) were transiently transfected into HeLa cells. Cell lysates were analyzed by Western blot (immunoblot) analysis. Cells were harvested 24 hour post-transfection with Versene (PBS, 1 mM EDTA, pH 8.0), and lysed on ice in lysis buffer (50 mM Tris pH 7.5/150 mM NaCl/1 mM EDTA/0.5% NP40/1 mM PMSF/10 mM NaF/1 mM sodium orthovanadate, protease inhibitors (Complete, Roche Diagnostics), and serine/threonine phosphatase inhibitors (Phosphatase Cocktail Set 1; Calbiochem, Inc.). Lysates were normalized for total protein using the Biorad Protein Assay Reagent. Proteins were fractionated by SDS-PAGE using 8% tris-glycine mini gels (INVITROGEN™) and transferred to PVDF membrane. Membranes were blocked in TBST (TBS/0.1% Tween 20) with 5% milk (blocking buffer) for 1 hour at room temperature on an orbital shaker and incubated with antibody (diluted to 2.5 micrograms/mL in blocking buffer) overnight at 4° C. Washes were performed at room temperature using TBST, followed by one hour incubation at room temperature with secondary antibody (donkey-anti-rabbit IgG, Horseradish Peroxidase linked F(ab′)2 fragment (Amersham Biosciences)) diluted 1:5000 in blocking buffer. Proteins were visualized with AMERSHAM ECL PLUS™ Western Blotting Detection Reagents (GE Healthcare Bio-Sciences Corp., Piscataway, N.J.).

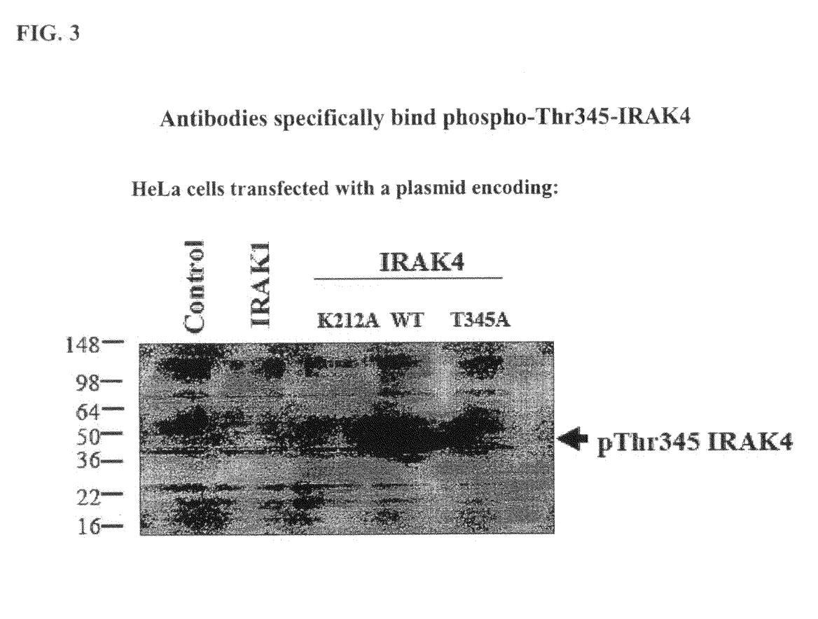

The polyclonal Phospho-Thr345-IRAK4 antibodies specifically detected IRAK4 in cell lysates from wild-type IRAK4, but did not produce a detectable signal from cell lysates with control vector or vectors encoding IRAK1, IRAK4-K211A, or IRAK4-T345A. See, FIG. 3.

### Example G

### Wild-Type IRAK4, but Not Kinase Inactive or IRAK4-T342A/T345A Mutants, Inhibits Signal Transduction Pathways Regulating Protein Synthesis

HeLa cells were transfected with firefly luciferase reporter constructs having inducible or constitutive promoters. HeLa cells were transfected with 0.3 micrograms of the reporter construct NFκB-firefly luciferase (Stratagene) along with 0.1 micrograms of SV40-Renilla luciferase (Promega) and 3 micrograms of either a control (empty) vector or expression vectors encoding wild-type IRAK4, kinase inactive IRAK4-K212A, IRAK4-T342A, TRAK4-T345A, or IRAK4-T342A/T345A.

Renilla luciferase expression is driven by an SV40 promoter. Production of luciferase from this vector signifies the effect of various IRAK4 molecules on the constitutive protein expression (i.e., not dependent on IL1-beta). Firefly luciferase driven by a NFκB promoter is inducible by IL1-beta. Luciferase production from this vector signifies the effects of various IRAK4 molecules on NFkB inducible protein expression.

Transfections were performed using LIPOFECTAMINE™ 2000 (INVITROGEN™). At 24 hours post-transfection cells were harvested in Versene (PBS/1 mM EDTA), centrifuged at approximately 200×G, resuspended in 500 microliters culture media, and counted. Transfectants were normalized for cell number/volume and seeded in triplicate into 96-well black and white isoplates (white bottom, black matrix plate) (WALLAC ISOPLATE™; PerkinElmer Life And Analytical Sciences, Inc., Wellesley, Mass.). Cells were treated with or without 10 nanograms/mL recombinant Human IL1β (IL1-beta; R&D Systems, Inc.) for 3 hours. Luciferase activity was detected using the Dual Glo Luciferase Assay System (Promega) following the manufacturer's instructions. Firefly and Renilla Luciferase levels were measured using a WALLAC MICROBETA® detector (PerkinElmer).

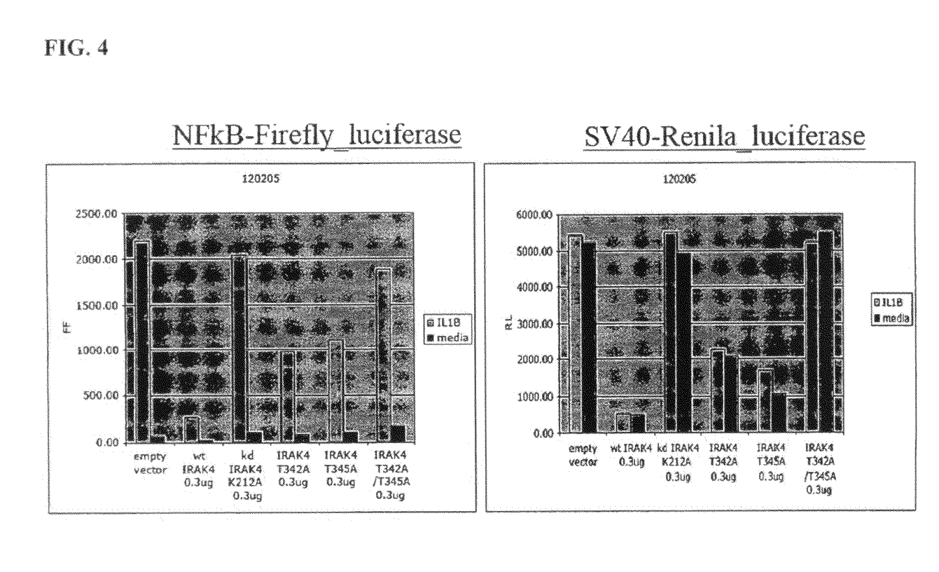

The luciferase reporter signals generated indicate that only wild-type IRAK4 effectively inhibited luciferase expression. In contrast, kinase inactive IRAK4 (IRAK4-K212A) and IRAK4-T342A/T345A were not effective (i.e., results were comparable to the vector only control). The single-threonine mutants (IRAK4-T342A and IRAK4-T345A) exhibited an intermediate level of activity in inhibiting protein synthesis. These results indicate that IRAK4 amino acid residues Thr-342 and Thr-345 are necessary for optimal IRAK4-induced inhibition of protein expression. See, FIG. 4.

### Example H

### Wild-Type IRAK4 (but Not Kinase Inactive or IRAK4-T342A/T345A Mutants) Induces Phosphorylation of EIF2alpha

HeLa cells were transfected using LIPOFECTAMINE™ 2000 (INVITROGEN™) with 3 micrograms of either a negative control vector or various IRAK4 expression constructs (i.e., vectors encoding wild-type IRAK4, kinase inactive (“kinase dead”) IRAK4-K212A, IRAK4-T342A, IRAK4-T345A, or IRAK4-T342A/T345A). Cells were harvested 24 hours post-transfection as previously described. Lysates were prepared for western blot (immunoblot) analysis as previously described. Proteins were fractionated on 10% tris-glycine mini gel (Invitrogen), and transferred onto PVDF membrane. After blocking for 1 hour in TBST/5% milk (blocking buffer), the membrane (blot) was incubated overnight with phospho-eIF2a-Ser52 polyclonal antibody (Biosource) diluted 1:500 in TBST/3% milk at 4° C. Membrane washes were performed with TBST at room temperature, followed by 1 hour incubation with the secondary antibody (donkey-anti-rabbit IgG, Horseradish Peroxidase linked F(ab′)2 fragment (Amersham Biosciences)) diluted 1:5000 in blocking buffer. Proteins were visualized with AMERSHAM ECL PLUS™ Western Blotting Detection Reagents (GE Healthcare Bio-Sciences Corp., Piscataway, N.J.).

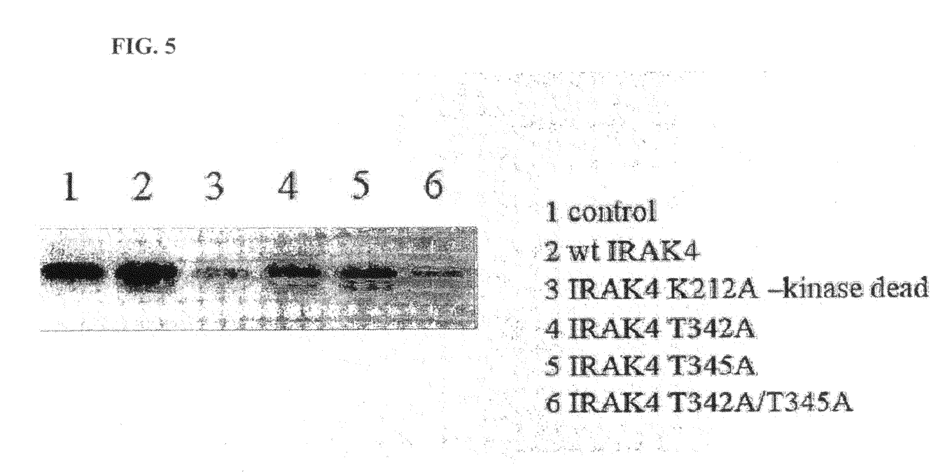

Results of this assay indicate that kinase active IRAK4, with both wild-type residue Thr342 and Thr345, is necessary to induce IRAK4 stimulated phosphorylation of EIF2alfpha at Ser51. See, FIG. 5.

### Example I

### Wild-Type IRAK4 (but Not Kinase Inactive or IRAK4-T342A/T345A Mutants) Inhibit IRAK1 and IκB (I-kappa-B) Phosphorylation

HeLa cells were transfected with 3 micrograms of each IRAK4 expression construct only, or with 2.5 micrograms of each IRAK4 expression construct in combination with 0.5 micrograms expression vector encoding wild-type IRAK1. As a negative control for IRAK1 phosphorylation, kinase dead IRAK1 was also transfected alone (2.5 micrograms vector only/0.5 micrograms vector encoding kinase dead IRAK1). Cells were harvested 24 hour post-transfection and lysates were prepared for western blotting (immunoblotting) as described previously. Proteins were fractionated on a 10% Tris-glycine mini gel. For western (immunoblot) analysis of IRAK1 (and GAPDH normalization control), 10 micrograms of total protein were loaded per lane. For analysis of IKB phosphorylation, 40 ug total protein was loaded per lane. After immunoblotting onto PVDF membrane, the blots were blocked overnight in TBST/5% milk at 4° C. Each blot was then incubated overnight at 4° C. with either IRAK1 Mab (IRAK1 (F-4) monoclonal IgG₁ antibody raised against amino acid residues 440-712; Santa Cruz) diluted 1:1000 in blocking buffer, or phospho-IκB-α (Ser32) polyclonal antibody (Cell Signaling Technology) diluted 1:500 in blocking buffer. For protein loading confirmation, the IRAK1 blot was incubated with a GAPDH monoclonal antibody diluted 1:200,000 in blocking buffer for 30 minutes at room temperature. Both blots were washed with TBST at room temperature and incubated at room temperature for 1 hour with secondary antibodies (sheep-anti-mouse IgG, Horseradish Peroxidase linked whole antibody (Amersham Biosciences) or donkey-anti-rabbit IgG, Horseradish Peroxidase linked F(ab′)2 fragment (Amersham Biosciences) respectively), diluted 1:5000 in TBST/5% milk. Proteins were visualized with AMERSHAM ECL PLUS™ Western Blotting Detection Reagents (GE Healthcare Bio-Sciences Corp., Piscataway, N.J.).

To confirm protein loading, the phospho-IKB blot was first stripped and reprobed. Confirmation of antibody stripping was performed with AMERSHAM ECL PLUS™ detection reagents. The blot was then blocked as before, and reprobed with a total IKB monoclonal antibody (IkB-a (112B2) monoclonal antibody; Cell Signaling Technology) diluted 1:500 in blocking buffer at 4° C. overnight. Secondary antibody incubation and antigen detection was performed as previously described for IRAK1 to assess the presence of IRAK1 (phosphorylated and unphosphorylated forms) and phosphorylated IκB (Phospho IκB) versus GAPDH as a normalization control.

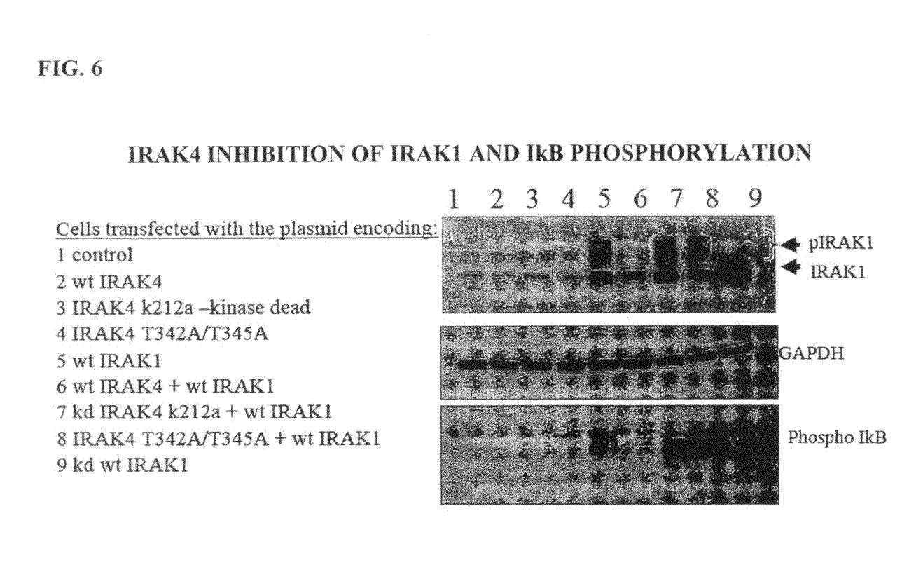

The results demonstrate that IRAK4 kinase activity is necessary to inhibit phosphorylation of IRAK1 and IRAK1-induced phosphorylation of IκB. See, FIG. 6. In addition to kinase activity, the results also demonstrate that Thr342 and Thr345 are critical for IRAK4-dependent inhibition of IRAK1 phosphorylation and for inhibiting IRAK1-induced phosphorylation of IκB. (The kinase inactive IRAK4-K212A and the IRAK4-T342A/T345A mutants did not inhibit phosphorylation of IRAK1 or IRAK1-induced phosphorylation of IκB.) See, FIG. 6. The results also demonstrate that IRAK4 does not induce IκB phosphorylation (NFκB activation). In contrast, IRAK1 does induce IκB phosphorylation, albeit in a manner independent of IRAK1 phosphorylation (IRAK1 kinase activity). See, FIG. 6.

### Example J

### Wild-Type IRAK4, but Not Kinase Inactive or IRAK4-T342A/T345A Mutants, Induces JNK1/2 Phosphorylation

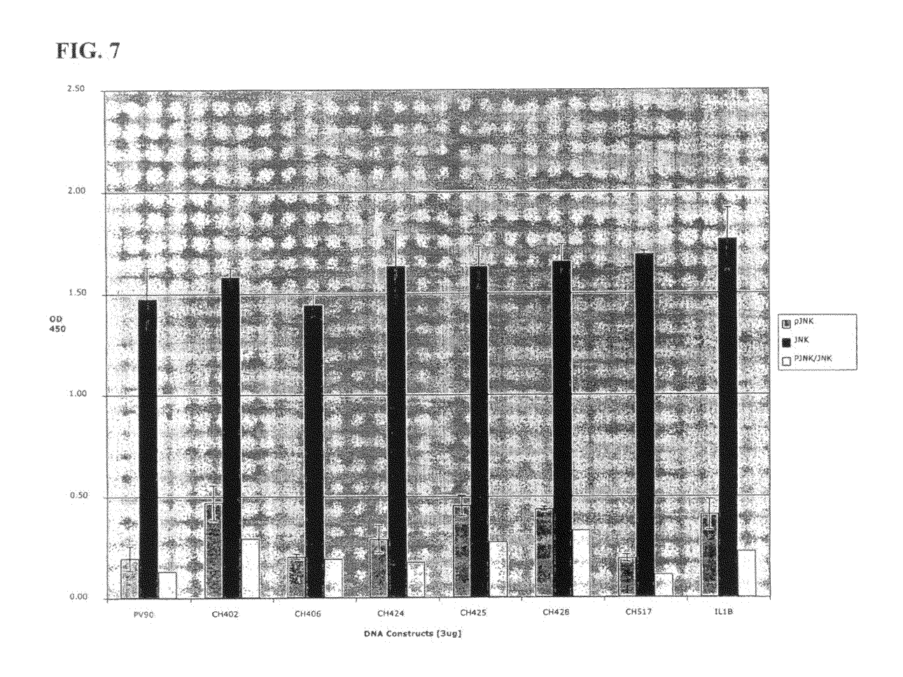

pJNK/totalJNK ELISA: HeLa cells were transfected with vectors encoding wild-type IRAK4, IRAK4 kinase domain mutants, or negative control vector, as described in preceding examples. For a positive control of JNK phosphorylation, cells were treated with 10 nanograms/mL IL1B for 15 minutes prior to harvesting. Cells were harvested 24 hour post transfection by washing with ice cold PBS then lysed by scraping into cell lysis buffer provided with the Pathscan phospho-SAPK/JNK (Thr183/TYR185) Sandwich Elisa kit (Cell Signaling Technology; 10× stock diluted to 1× and supplemented with 1 mM PMSF, 10 mM NaF, 1 mM sodium orthovanadate, protease inhibitors (Complete, Roche Diagnostics), and serine/threonine phosphatase inhibitors (Phosphatase Cocktail Set 1, Calbiochem). Total protein concentration was determined using the BioRad Protein Dye Reagent. Samples were normalized for protein concentration and diluted in sample dilution buffer provided with the Sandwich Elisa kit. Using both the Pathscan phospho-SAPK/JNK (Thr183/TYR185) Sandwich Elisa kit, and the Pathscan total SAPK/JNK Sandwich Elisa kit, samples were loaded in duplicate (at 100 microliters/well sample volume) on the ELISA strips provided. The ELISA strips were incubated overnight at 4° C., and the assay was developed following the manufacturers instructions with the reagents provided. The level of phospho-JNK, and total JNK was measured using a Spectramax M5 plate reader (Molecular Devices) at 450 nm. See, FIG. 7.

### Example K

### Analysis of Signal Transduction Pathways Activated by IRAK4 Kinase Active, IRAK4 Kinase Inactive, IRAK1 Kinase Active, and IRAK1 Kinase Inactive Proteins

Affymetrix microarrays U96a (Affymetrix, Inc., Santa Clara, Calif., U.S.A.) were used to determine gene expression profiles induced in cells 24 hours following overexpression of IRAK4 and IRAK1, kinase active and kinase inactive proteins. Results of these experiments (data not shown) indicated:

Wild-type IRAK4 (but not kinase inactive IRAK4) induces expression of genes under JNK pathway control, but does not induce expression of genes under NFkB or p38 control.

IRAK1 kinase activity (i.e., wild-type IRAK1) is only necessary to induce expression of a subset of genes induced by IRAK1 (i.e., gene expression induced by kinase active and kinase inactive IRAK1).

Both wild-type IRAK1 and kinase inactive IRAK1 induce expression of genes under NFkB, p38, JNK and IRF signal transduction control pathways.

### INCORPORATION BY REFERENCE

U.S. Provisional Patent Application No. 60/881,190 filed on Jan. 19, 2007 is hereby incorporated by reference in its entirety.

What is claimed is:

1. An isolated antibody or antigen-binding fragment thereof that specifically binds to an IRAK4 epitope comprising phosphorylated threonine 342 (Phospho-Thr342).

2. The antibody or fragment thereof of claim 1, wherein the IRAK4 epitope is human IRAK4.

3. The antibody or fragment thereof of claim 1, wherein the antibody is a polyclonal antibody.

4. The antibody or fragment thereof of claim 1, wherein the antibody is a monoclonal antibody.

5. The antibody or fragment thereof of claim 1, wherein the antibody is humanized.

6. The antibody or fragment thereof of claim 1, wherein the IRAK4 epitope comprises SEQ ID NO:4.

7. An isolated antibody or antigen-binding fragment thereof that specifically binds to an IRAK4 epitope comprising phosphorylated threonine 345 (Phospho-Thr345).

8. The antibody or fragment thereof of claim 7, wherein the IRAK4 epitope is human IRAK4.

9. The antibody or fragment thereof of claim 7, wherein the antibody is a polyclonal antibody.

10. The antibody or fragment thereof of claim 7, wherein the antibody is a monoclonal antibody.

11. The antibody or fragment thereof of claim 7, wherein the antibody is a humanized antibody.

12. The antibody or fragment thereof of claim 7, wherein the IRAK4 epitope comprises SEQ ID NO:4.

13. An isolated antibody or antigen-binding fragment thereof that specifically binds to an IRAK4 epitope comprising phosphorylated serine 346 (Phospho-Ser346).

14. The antibody or fragment thereof of claim 13, wherein the IRAK4 epitope is human IRAK4.

15. The antibody or fragment thereof of claim 13, wherein the antibody is a polyclonal antibody.

16. The antibody or fragment thereof of claim 13, wherein the antibody is a monoclonal antibody.

17. The antibody or fragment thereof of claim 13, wherein the antibody is a humanized antibody.

18. The antibody or fragment thereof of claim 13, wherein the IRAK4 epitope comprises SEQ ID NO:4.

19. An isolated antibody that specifically binds to the same epitope as the antibody secreted by a hybridoma cell line selected from the group consisting of:
(a) 101C2;
(b) 102C4;
(c) 103B6; and,
(d) 142B2.

20. An isolated antibody or antigen-binding fragment thereof that specifically binds to IRAK-4 phosphorylated at threonine 342 (Phospho-Thr342), threonine 345 (Phospho-Thr345) or serine 346 (Phospho-Ser346), wherein said antibody or fragment thereof competitively inhibits an antibody secreted by a hybridoma cell line selected from the group consisting of 101C2, 102C4, 103B6, and 142B2.

21. An isolated antibody secreted by a hybridoma cell line selected from the group consisting of 101C2, 102C4, 103B6, and 142B2.

22. A hybridoma cell line selected from the group consisting of 101C2, 102C4, 103B6, and 142B2.

23. A method for determining if a test agent inhibits human IRAK-4 kinase activity, said method comprising:
(a) subjecting a first and a second cell that each express a human IRAK-4 or a fragment of human IRAK-4 comprising the IRAK-4 kinase domain to conditions in which human IRAK-4 is normally activated;
(b) contacting said first cell with a test agent;
(c) liberating said human IRAK-4 or fragment thereof from said first and second cells;
(d) adding a phospho-IRAK-4-specific antibody to said liberated human IRAK-4 or fragment thereof obtained from said first and second cells; and,
(e) determining if said phospho-IRAK-4-specific antibody binds to liberated human IRAK-4 or fragment thereof obtained from said first and second cells;
wherein said phospho-IRAK-4-specific antibody is selected from the group consisting of: (i) an antibody or antigen-binding fragment thereof that specifically binds to an IRAK4 epitope comprising phosphorylated threonine 342 (Phospho-Thr342); (ii) an antibody or antigen-binding fragment thereof that specifically binds to an IRAK4 epitope comprising phosphorylated threonine 345 (Phospho-Thr345); and (iii) an antibody or antigen-binding fragment thereof that specifically binds to an IRAK4 epitope comprising phosphorylated serine 346 (Phospho-Ser346); and,
wherein reduced binding of said phospho-IRAK-4-specific antibody to liberated human IRAK-4 or fragment thereof obtained from said first cell as compared to the binding of said phospho-IRAK-4-specific antibody to human IRAK-4 or fragment thereof liberated from said second cell indicates that the test agent inhibits human IRAK-4 kinase activity.

24. The method of claim 23, wherein said test agent is contacted with said first cell prior to or simultaneous with subjecting said first cell to conditions in which human IRAK-4 is normally activated.

25. A method for determining if a test agent inhibits human IRAK-4 kinase activity, said method comprising:
(a) subjecting a first and second cell-free system, each comprising a human IRAK-4 or a fragment of human IRAK-4 comprising the IRAK-4 kinase domain, to conditions in which human IRAK-4 is normally activated;
(b) adding to said first cell-free system a test agent;
(c) adding to said first and second cell-free systems a phospho-IRAK-4-specific antibody; and,
(d) determining if said phospho-IRAK-4-specific antibody binds to said human IRAK-4 or fragment thereof in said first and second cell-free systems;
wherein said phospho-IRAK-4-specific antibody is selected from the group consisting of: (i) an antibody or antigen-binding fragment thereof that specifically binds to an IRAK4 epitope comprising phosphorylated threonine 342 (Phospho-Thr342); (ii) an antibody or antigen-binding fragment thereof that specifically binds to an IRAK4 epitope comprising phosphorylated threonine 345 (Phospho-Thr345); and (iii) an antibody or antigen-binding fragment thereof that specifically binds to an IRAK4 epitope comprising phosphorylated serine 346 (Phospho-Ser346); and,
wherein reduced binding of said phospho-IRAK-4-specific antibody to human IRAK-4 or fragment thereof in said first cell-free system as compared to the binding of said phospho-IRAK-4-specific antibody to human IRAK-4 or fragment thereof in said second cell-free system indicates that the test agent inhibits human IRAK-4 kinase activity.

26. The method of claim 25, wherein said test agent is added to said first cell-free system prior to or simultaneous with subjecting said first cell-free system to conditions in which human IRAK-4 is normally activated.

27. A method for identifying a condition characterized by activation of the IRAK pathway, said method comprising:
(a) contacting a first tissue sample with a phospho-IRAK-4-specific antibody, wherein said first tissue sample is obtained from a first subject suspected of having a condition characterized by activation of the IRAK pathway;
(b) contacting a second tissue sample with said phospho-IRAK-4-specific antibody, wherein said second tissue sample is obtained from a second subject known not to have said condition; and
(c) determining if said phospho-IRAK-4-specific antibody binds to human IRAK-4 in said first and second samples;
wherein said phospho-IRAK-4-specific antibody is selected from the group consisting of: (i) an antibody or antigen-binding fragment thereof that specifically binds to an IRAK4 epitope comprising phosphorylated threonine 342 (Phospho-Thr342); (ii) an antibody or antigen-binding fragment thereof that specifically binds to an IRAK4 epitope comprising phosphorylated threonine 345 (Phospho-Thr345); and (iii) an antibody or antigen-binding fragment thereof that specifically binds to an IRAK4 epitope comprising phosphorylated serine 346 (Phospho-Ser346); and
wherein reduced binding of said phospho-IRAK-4-specific antibody to said first sample as compared to binding of said phospho-IRAK-4-specific antibody to said second sample indicates that said first subject has a condition characterized by activation of the IRAK pathway.

28. The method of claim 27, wherein said phospho-IRAK-4-specific antibody is an antibody or antigen-binding fragment thereof that specifically binds to human IRAK-4 phosphorylated at Thr342.

29. The method of claim 27, wherein said phospho-IRAK-4-specific antibody is an antibody or antigen-binding fragment thereof that specifically binds to human IRAK-4 phosphorylated at Thr345.

30. The method of claim 27, wherein said phospho-IRAK-4-specific antibody is an antibody or antigen-binding fragment thereof that specifically binds to human IRAK-4 phosphorylated at Ser346.

31. The method of claim 27, wherein said phospho-IRAK-4-specific antibody is an antibody or antigen-binding fragment thereof that specifically binds to human IRAK-4 phosphorylated at both Thr342 and Thr345.

32. The method of claim 27, wherein said phospho-IRAK-4-specific antibody is an antibody or antigen-binding fragment thereof that specifically binds to human IRAK-4 phosphorylated at both Thr342 and Ser346.

33. The method of claim 27, wherein said phospho-IRAK-4-specific antibody is an antibody or antigen-binding fragment thereof that specifically binds to human IRAK-4 phosphorylated at both Thr345 and Ser346.

34. The method of claim 27, wherein said phospho-IRAK-4-specific antibody is an antibody or antigen-binding fragment thereof that specifically binds to human IRAK-4 phosphorylated at Thr342, Thr345, and Ser346.

35. The method of claim 27, wherein the antibody is a polyclonal antibody.

36. The method of claim 27, wherein the antibody is a monoclonal antibody.

37. The method of claim 27, wherein the antibody is a humanized antibody.

38. The method of claim 27, wherein said condition characterized by activation of the IRAK4 pathway is a pulmonary disease, an autoimmune disease, a cancer, a cardiovascular disease, a neurodegenerative disease, sepsis, osteoarthritis, osteoporosis, a bacterial infection, a viral infection, a skin disease, or an inflammatory disease.

39. The method of claim 38, wherein said condition is an autoimmune disease.

40. The method of claim 38, wherein said autoimmune disease is selected from the group consisting of rheumatoid arthritis, multiple sclerosis, lupus erithrematosis, inflammatory bowel disease, ulcerative colitis, and psoriasis.

41. The method of claim 38, wherein said condition is a bacterial infection.

42. The method of claim 41, wherein said bacterial infection is Staphylococcus spp., Salmonella spp., Shigellae spp., Yersiniae spp., Bacillus spp., Neisseria spp., or anthrax infection.

43. The method of claim 42, wherein said bacterial infection is Staphylococcus pneumonia infection.

44. The method of claim 42, wherein said bacterial infection is Staphylococcus aureus infection.

45. The method of claim 38, wherein said viral infection is influenza or HIV.


SEQUENCE LISTING (23 sequences)

SEQ ID NO: 1 - protein, 298 aa, Homo sapiens
TRFHSFSFYELKNVTNNFDERPISVGGNKMGEGGFGVVYKGYVNNTTVAVKKLAAMVDITTEELKQQFDQEIKVMAKCQHENLVELLGFSSDGDDLCLVYVYMPNGSLLDRLSCLDGTPPLSWHMRCKIAQGAANGINFLHENHHIHRDIKSANILLDEAFTAKISDFGLARASEKFAQTVMTSRIVGTTAYMAPEALRGEITPKSDIYSFGVVLLEIITGLPAVDEHREPQLLLDIKEEIEDEEKTIEDYIDKKMNDADSTSVEAMYSGASQCRHEKKNKSPDIKKVHQLLQEMTAS

SEQ ID NO: 2 - protein, 460 aa, Homo sapiens
MNKPITPSTYVRCLNVGLIRKLSDFIDPQEGWKKLAVAIKKPSGDDRYNQFHIRRFEALLQTGKSPTSELLFDWGTTNCTVGDLVDLLIQNEFFAPASLLLPDAVPKTANTLPSKEAITVQQKQMPFCDKDRTLMTPVQNLEQSYMPPDSSSPENKSLEVSDTRFHSFSFYELKNVTNNFDERPISVGGNKMGEGGFGVVYKGYVNNTTVAVKKLAAMVDITTEELKQQFDQEIKVMAKCQHENLVELLGFSSDGDDLCLVYVYMPNGSLLDRLSCLDGTPPLSWHMRCKIAQGAANGINFLHENHHIHRDIKSANILLDEAFTAKISDFGLARASEKFAQTVMTSRIVGTTAYMAPEALRGEITPKSDIYSFGVVLLEIITGLPAVDEHREPQLLLDIKEEIEDEEKTIEDYIDKKMNDADSTSVEAMYSGASQCRHEKKNKSPDIKKVHQLLQEMTAS

SEQ ID NO: 3 - protein, 16 aa, Artificial Sequence
  Synthetic peptide for use as an antigen
CKFAQTVMTSRIVGTT

SEQ ID NO: 4 - protein, 15 aa, Artificial Sequence
  Synthetic peptide for use as an antigen
KFAQTVMTSRIVGTT

SEQ ID NO: 5 - protein, 16 aa, Artificial Sequence
  Synthetic peptide for use as an antigen
CKFAQTVMTSRIVGTT

SEQ ID NO: 6 - protein, 15 aa, Artificial Sequence
  Synthetic peptide for use as an antigen
KFAQTVMTSRIVGTT

SEQ ID NO: 7 - DNA, 43 bp, Artificial sequence
  Synthetic oligonucleotide for use as PCR primer
ataagaatgcggccgccatgaacaaacccataacaccatcaac

SEQ ID NO: 8 - DNA, 38 bp, Artificial sequence
  Synthetic oligonucleotide for use as PCR primer
atagtttagcggccgcttaagaagctgtcatctcttgc

SEQ ID NO: 9 - DNA, 33 bp, Artificial sequence
  Synthetic oligonucleotide for use as PCR primer
gagaagtttgcccaggcagtcatgactagcaga

SEQ ID NO: 10 - DNA, 33 bp, Artificial Sequence
  Synthetic oligonucleotide for use as PCR primer
tctgctagtcatgactgcctgggcaaacttctc

SEQ ID NO: 11 - DNA, 29 bp, Artificial sequence
  Synthetic oligonucleotide for use as PCR primer
ccagacagtcatggctagcagaattgtgg

SEQ ID NO: 12 - DNA, 29 bp, Artificial sequence
  Synthetic oligonucleotide for use as PCR primer
ccacaattctgctagccatgactgtctgg

SEQ ID NO: 13 - DNA, 41 bp, Artificial sequence
  Synthetic oligonucleotide for use as PCR primer
gcccagacagtcatgactgcaagaattgtgggaacaacagc

SEQ ID NO: 14 - DNA, 41 bp, Artificial sequence
  Synthetic oligonucleotide for use as PCR primer
gctgttgttcccacaattcttgcagtcatgactgtctgggc

SEQ ID NO: 15 - DNA, 28 bp, Artificial sequence
  Synthetic oligonucleotide for use as PCR primer
ccaggcagtcatggctagcagaattgtg

SEQ ID NO: 16 - DNA, 28 bp, Artificial sequence
  Synthetic oligonucleotide for use as PCR primer
cacaattctgctagccatgactgcctgg

SEQ ID NO: 17 - DNA, 42 bp, Artificial sequence
  Synthetic oligonucleotide for use as PCR primer
cttctgagaagtttgcccaggccgtcatgactgcaagaattg

SEQ ID NO: 18 - DNA, 42 bp, Artificial sequence
  Synthetic oligonucleotide for use as PCR primer
caattcttgcagtcatgacggcctgggcaaacttctcagaag

SEQ ID NO: 19 - DNA, 32 bp, Artificial sequence
  Synthetic oligonucleotide for use as PCR primer
gcccagacagtcatggccgcaagaattgtggg

SEQ ID NO: 20 - DNA, 32 bp, Artificial sequence
  Synthetic oligonucleotide for use as PCR primer
cccacaattcttgcggccatgactgtctgggc

SEQ ID NO: 21 - DNA, 23 bp, Artificial sequence
  Synthetic oligonucleotide for use as PCR primer
gtggcagtggcggcgcttgcagc

SEQ ID NO: 22 - DNA, 23 bp, Artificial sequence
  Synthetic oligonucleotide for use as PCR primer
gctgcaagcgccgccactgccac

SEQ ID NO: 23 - protein, 16 aa, Artificial Sequence
  Synthetic peptide for use as an antigen
CKFAQTVMTSRIVGTT